(19) United States
(12) Patent Application Publication
(10) Pub. No.: US 2010/0248348 A1
(43) Pub. Date: Sep. 30, 2010

(54) TARGET NUCLEIC ACID MEASURING APPARATUS

(75) Inventors: Kaneyasu Ohkawa, Sagamihara-shi (JP); Yoshinobu Akimoto, Tokyo (JP); Mari Nakamoto, Tokyo (JP); Takeshi Wada, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: OLYMPUS CORPORATION, Tokyo (JP)

(21) Appl. No.: 12/692,059

(22) Filed: Jan. 22, 2010

(30) Foreign Application Priority Data
Mar. 27, 2009 (JP) 2009-080659
Mar. 27, 2009 (JP) 2009-080660
Mar. 27, 2009 (JP) 2009-080661

(51) Int. Cl. C12M 1/34

(52) U.S. Cl. 435/287.2

(57) ABSTRACT

The present invention relates to calculation of an amount equivalent to an initial template amount in a test sample by fitting a theoretical expression to an amount equivalent to an amplification amount of target nucleic acid for each thermal cycle number, wherein the theoretical expression includes an environmental coefficient as an exponent parameter and a parameter of the amount equivalent to the initial template amount, and includes at least one parameter among an amount equivalent to a saturation amount upon the target nucleic acid amplification, a reaction acceleration coefficient, and a reaction inhibition coefficient.

### CROSS-REFERENCE TO RELATED APPLICATIONS

This application is based upon and claims the benefit of priority from Japanese Patent Applications No. 2009-080659, No. 2009-080660, and No. 2009-080661, each filed Mar. 27, 2009, the entire contents of which are incorporated herein by reference.

### BACKGROUND OF THE INVENTION

1. Field of the Invention

The present invention relates to a target nucleic acid measuring method, a target nucleic acid measuring apparatus, a target nucleic acid measuring system, and a target nucleic acid measuring program.

2. Description of the Related Art

There has been conventionally reported a nucleic acid amplification reaction called polymerase chain reaction (hereinafter, described as “PCR”). The PCR is capable of amplifying a specific nucleic acid region in a nucleic acid molecule, even to about one million times the original amount within a vessel. By utilizing the PCR, it is possible to detect a specific nucleic acid region of target nucleic acid in an amplified state, and in terms of sensitivity, it is even possible to detect a pathogen from the nucleic acid of one molecule.

As a general method for detecting this amplified specific nucleic acid region, there is available a method of separating the reaction solution obtained after completion of a PCR using agarose electrophoresis, subsequently staining the nucleic acids, and discriminating the specific nucleic acid region based on the degree of mobility of the band (molecular weight); or a method of detecting this reaction solution by a dot hybridization method.

Here, a method for measuring the initial template amount is disclosed in JP-A-7-163397. In this method of measurement, first, a plurality of test samples are prepared such that one test sample has a specific nucleic acid sequence at an unknown concentration, while other test samples contain the same specific nucleic acid sequence at different known concentrations. Then, the test samples of known concentrations and the test sample of unknown concentration are subjected to a thermal cycle concurrently in multiple cycles. Subsequently, the fluorescence radiated from the test samples is measured, and the number of cycles required for each of the reaction mixtures to emit fluorescence at a certain intensity (for example, a predetermined intensity at or above the detection level) in real time (hereinafter, referred to as “C_{T} value”) is determined. Then, a standard curve plotting the concentration of the specific nucleic acid sequence versus the C_{T} value is produced for the test samples of known concentrations, and the C_{T} value of the test sample of unknown nucleic acid concentration is assigned into the produced standard curve, to thereby determine the amount of initial template of the specific nucleic acid sequence in the test sample of unknown concentration.

The product manual of the Real-Time PCR System (trade name) manufactured by Applied Biosystems, Inc. discloses a comparative C_{T} method, which is a relative quantification method of determining a relative value from the difference between C_{T} values of a test sample and a reference sample (see URL: http://www.appliedbiosystems.co.jp/website/jp/biobeat/contents.jsp?COLUMNPGCD=78973&COLUMNCD=76448&TYPE=C&BIOCATEGORYC D=7). In this method, a relative value is determined from the difference between the C_{T} values of the test sample and the reference sample, and the initial template amount is determined under an assumption that the PCR amplification efficiency is 100%.

JP-A-8-66199 also discloses a method for measuring the initial template amount as follows. In this measuring method, first, a test sample is subjected to thermal cycles in a plurality of cycles. At this time, since fluorescence is radiated from the test sample at an intensity equivalent to the amplification amount of the nucleic acid, this radiated fluorescence is measured. Then, the measured value is converted to a molar concentration value of dsDNA, and a measurement curve plotting the molar concentration of dsDNA versus the cycle number is generated. A theoretical curve that takes the starting molar concentration of the primer as one of the parameters is fitted to the measurement curve, and thus the molar concentration of dsDNA at cycle number 0, that is, the initial template amount in the test sample, is determined.

### SUMMARY OF THE INVENTION

A target nucleic acid measuring apparatus according to one aspect of the present invention includes a storage unit and a control unit. The storage unit includes an amplification amount equivalent storage unit that stores an amount equivalent to an amplification amount of target nucleic acid corresponding to thermal cycle number, and a theoretical expression storage unit that stores a theoretical expression. The theoretical expression includes an environmental coefficient as an exponent parameter and a parameter of an amount equivalent to an initial template amount in the test sample, and includes at least one parameter among an amount equivalent to a saturation amount upon the target nucleic acid amplification, a reaction acceleration coefficient, and a reaction inhibition coefficient. The control unit includes a theoretical expression fitting unit that fits the theoretical expression to the amount equivalent to the amplification amount of the target nucleic acid for each thermal cycle number stored in the storage unit. The control unit further includes an initial template amount equivalent calculating unit that calculates the amount equivalent to the initial template amount from the theoretical expression fitted by the theoretical expression fitting unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a flowchart for explaining an overview of Embodiment 1;

FIG. 2 is a block diagram showing an example of a configuration of a target nucleic acid measuring apparatus 100 according to Embodiment 1 to which the present invention is applied;

FIG. 3 is a flowchart showing an example of the processing of the target nucleic acid measuring apparatus 100 according to Embodiment 1;

FIG. 4 is a diagram showing measurement data obtained by the measuring apparatus ABI7900 (trade name) manufactured by Applied Biosystems, Inc.;

FIG. 5 is a diagram in which thermal cycle number n is shown on the horizontal axis, and an increasing rate Δₙ is shown on the vertical axis;

FIG. 6 is a diagram showing comparison between the actual measurement and the theoretical calculation, pulling out the region to be fitted that satisfies the relational expression “0≦Δₙ≦ρ≦1” from FIG. 5;

FIG. 7 is a diagram showing the measurement result of the amplification curve, which is the result of cycle number versus fluorescence intensity in each of the test samples;

FIG. 8 is a diagram showing conditions of the theoretical expression;

FIG. 9 is a diagram showing the calculation result of the template amount equivalent N₀ according to Example 2;

FIG. 10 is a diagram showing the theoretical calculation values for the test samples No. 1 to No. 5, obtained on the condition 4;

FIG. 11 is a diagram showing results of fitting the theoretical expression to measurement data using values of ρ and μ obtained by fitting the theoretical expression for standard sample having a known template amount after determining a value of the environmental coefficient K within the scope of K=1.0 to 1.7;

FIG. 12 is a diagram showing results of fitting the theoretical expression to measurement data using values of ρ and μ obtained by fitting the theoretical expression for standard sample having a known template amount after determining a value of the environmental coefficient K within the scope of K=1.0 to 1.7;

FIG. 13 is a diagram showing results of fitting the theoretical expression to measurement data using values of ρ and μ obtained by fitting the theoretical expression for standard sample having a known template amount after determining a value of the environmental coefficient K within the scope of K=1.0 to 1.7;

FIG. 14 is a diagram showing results of fitting the theoretical expression to measurement data using values of ρ and μ obtained by fitting the theoretical expression for standard sample having a known template amount after determining a value of the environmental coefficient K within the scope of K=1.0 to 1.7;

FIG. 15 is a diagram showing a ratio of the initial template amount equivalent N₀ calculated using the measurement data of Experiment 1 in which 2000 pg/μl and 500 pg/μl of the initial template amounts were prepared;

FIG. 16 is a diagram showing a ratio of the initial template amount equivalent N₀ calculated using the measurement data of Experiment 2 in which 2000 pg/μl and 500 pg/μl of the initial template amounts were prepared;

FIG. 17 is a diagram showing a ratio of the initial template amount equivalent N₀ calculated using the measurement data of Experiment 3 in which 2000 pg/μl and 500 pg/μl of the initial template amounts were prepared;

FIG. 18 is a flowchart for explaining an overview of Embodiment 2;

FIG. 19 is a block diagram showing an example of a configuration of a target nucleic acid measuring apparatus 100 according to Embodiment 2 to which the present invention is applied;

FIG. 20 is a flowchart showing an example of the processing of the target nucleic acid measuring apparatus 100 according to Embodiment 2;

FIG. 21 is a diagram showing a relationship between amplification product concentration and fluorescence intensity;

FIG. 22 is a diagram showing calculation results of the parameters for the test samples No. 1 and No. 2;

FIG. 23 is a diagram showing the calculation result of the initial template amount;

FIG. 24 is a diagram showing the standard curve of the Ct value versus the initial template amount;

FIG. 25 is a diagram showing a calculation result of the initial template amount using the standard curve of Ct value;

FIG. 26 is a diagram showing comparison between the calculation results of the initial template amounts according to Example 4-1 and the conventional example;

FIG. 27 is a diagram showing a relationship between molar concentration of FAM molecule and fluorescence intensity;

FIG. 28 is a diagram showing the calculation result of the parameters for the test samples No. 3 and No. 4;

FIG. 29 is a diagram showing the calculation result of the initial template amount;

FIG. 30 is a diagram showing a relationship between amplification product amount (template amount) and fluorescence intensity;

FIG. 31 is a diagram showing calculation results of the parameters for the test samples No. 5 and No. 6;

FIG. 32 is a diagram showing the calculation result of the initial template amount;

FIG. 33 is a diagram showing the standard curve of the Ct value versus the initial template amount;

FIG. 34 is a diagram showing a calculation result of the initial template amount using the standard curve of Ct value as the conventional example;

FIG. 35 is a diagram showing comparison between the calculation results of the initial template amounts according to Example 4-3 and the conventional example;

FIG. 36 is a flowchart for explaining an overview of Embodiment 3;

FIG. 37 is a block diagram showing an example of a configuration of a target nucleic acid measuring apparatus 100 according to Embodiment 3 to which the present invention is applied;

FIG. 38 is a flowchart showing an example of the processing of the target nucleic acid measuring apparatus 100 according to Embodiment 3;

FIG. 39 is a flowchart explaining a measuring method of the target nucleic acid according to Example 5;

FIG. 40 is a diagram showing the standard curve of the Ct value versus the initial template amount;

FIG. 41 is a diagram showing the calculation result according to the conventional examples, which are the standard curve method and the comparative Ct method;

FIG. 42 is a diagram showing the calculation result of each of the parameters by fitting in the 2 cases of Condition 1 and Condition 2 for the test samples No. 1 and No. 2;

FIG. 43 is a diagram showing the initial template amount (equivalent) and the initial template amount ratio calculated according to Example 5-1 and the conventional example;

FIG. 44 is a diagram showing a value of each of the calculated parameters;

FIG. 45 is a diagram showing the initial template amount calculated for the test samples No. 1 and No. 2; and

FIG. 46 is a diagram showing a result of the comparison of the calculated initial template amount between Example 5-2 and the conventional method (standard curve method) for the test samples No. 1 and No. 2.

### DETAILED DESCRIPTION OF THE PREFERRED EMBODIMENTS

Hereinafter, exemplary embodiments of a target nucleic acid measuring method, a target nucleic acid measuring apparatus, a target nucleic acid measuring system, and a target nucleic acid measuring program according to the invention will be explained in detail with reference to the accompanying drawings. The invention is not limited to the embodiments.

### Embodiment 1

### Overview of Embodiment 1

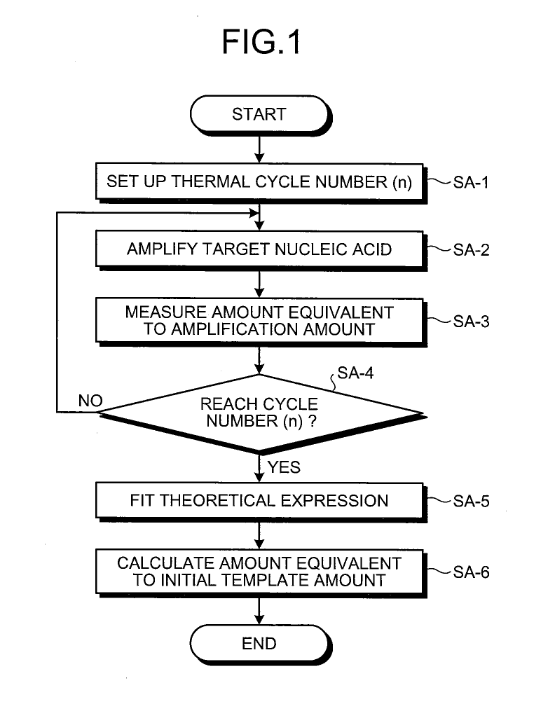

Hereinafter, an overview of Embodiment 1 will be explained with reference to FIG. 1, and then a configuration and processing of Embodiment 1 will be explained in detail. FIG. 1 is a flowchart for explaining an overview of Embodiment 1.

As shown in FIG. 1, according to Embodiment 1, first, the thermal cycle number (n) of a nucleic acid amplification reaction is set up (step SA-1). That is, the thermal cycle number (n) is set up for repeatedly performing the nucleic acid amplification reaction.

According to Embodiment 1, the nucleic acid amplification reaction of the test sample is performed to amplify the target nucleic acid (step SA-2).

According to Embodiment 1, an amount equivalent to an amplification amount of the target nucleic acid (hereinafter, referred to as “amplification amount equivalent”) is measured corresponding to the thermal cycle number (step SA-3). Here, the “amplification amount” means the total amount of the target nucleic acid amplified from thermal cycle number 1 to the thermal cycle number when measuring. Also, the “amplification amount equivalent” is an amount equivalent to the amplification amount of the target nucleic acid, obtained by an arbitrary label. For example, the “amplification amount equivalent” is a signal intensity detected from an intercalator that emits the signal in the presence of a double-strand DNA, or a signal intensity detected from a reporter molecule that emits the signal according to an extension reaction during the amplification reaction.

According to Embodiment 1, it is determined whether current thermal cycle number has reached the set thermal cycle number (n) (step SA-4), and when the current thermal cycle number has not reached the set thermal cycle number (n) (step SA-4, No), the process is returned to the step SA-2. On the other hand, when the current thermal cycle number has reached the set thermal cycle number (n) (step SA-4, Yes), the nucleic acid amplification reaction and the measurement of the amplification amount equivalent according the reaction are finished.

According to Embodiment 1, a theoretical expression is fitted to the amplification amount equivalent measured in each thermal cycle (step SA-5). The theoretical expression according to Embodiment 1 includes an environmental coefficient as an exponent parameter, and a parameter of an amount equivalent to an initial template amount in the test sample (hereinafter, referred to as “initial template amount equivalent”). Further, this theoretical expression includes at least one parameter among an amount equivalent to a saturation amount upon the target nucleic acid amplification, a reaction acceleration coefficient, and a reaction inhibition coefficient. That is, according to Embodiment 1, the parameters of the theoretical expression are determined in this step SA-5 so as to fit the theoretical expression to the amplification amount equivalent measured in each thermal cycle.

In the theoretical expression, an amplification efficiency of the nucleic acid amplification reaction may be defined by the environmental coefficient and at least one parameter among the amount equivalent to the saturation amount upon the target nucleic acid amplification, the reaction acceleration coefficient, and the reaction inhibition coefficient. The theoretical expression may express that the amplification amount equivalent is equal to the product of the initial template amount equivalent and a sequence of the amplification efficiencies to each of which 1 is added for each thermal cycle number. According to Embodiment 1, the theoretical expression may be fitted using the least square method, or at least one of the parameters may be a fixed value.

According to Embodiment 1, the initial template amount equivalent is calculated from the theoretical expression fitted to the amplification amount equivalent (step SA-6). For example, according to Embodiment 1, the initial template amount equivalent is calculated based on the theoretical expression in which the parameters have been determined by fitting.

An overview of Embodiment 1 has been explained hereinbefore. According to Embodiment 1, the initial template amount equivalent can be precisely determined using a theoretical expression that allows to be fitted directly to the measured amplification amount equivalent and is capable of precisely reflecting the actual amplification efficiency.

Configuration of Target Nucleic Acid Measuring Apparatus

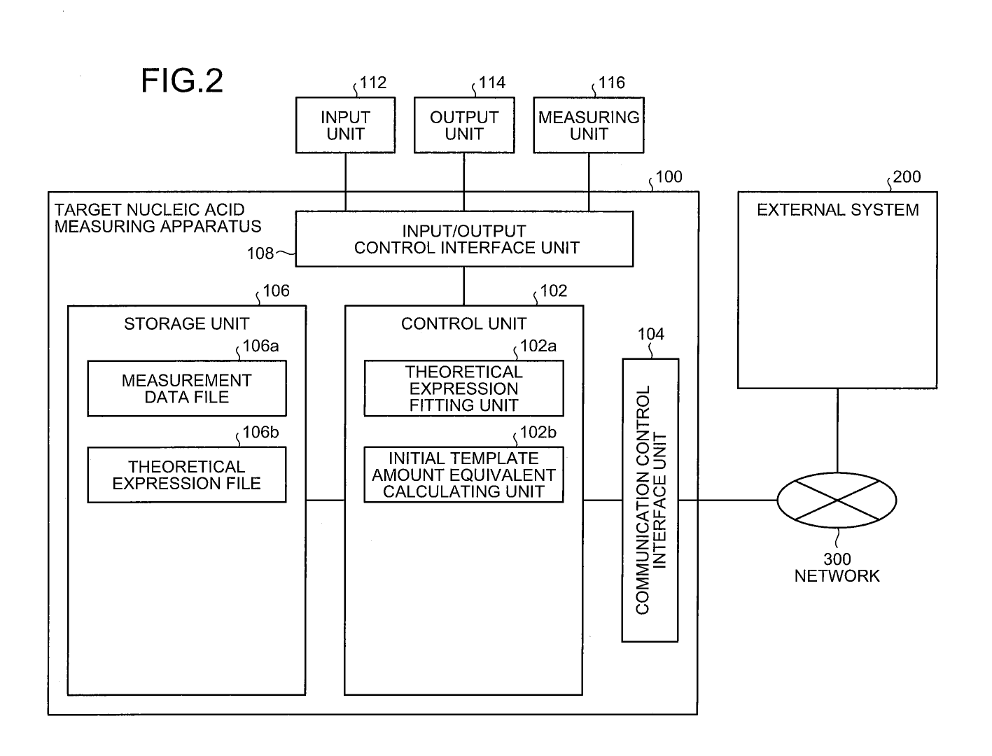

Next, a configuration of a target nucleic acid measuring apparatus according to Embodiment 1 will be explained below with reference to FIG. 2. FIG. 2 is a block diagram showing an example of a configuration of a target nucleic acid measuring apparatus 100 according to Embodiment 1 to which the present invention is applied, and schematically depicts only a part related to Embodiment 1 in the configuration.

In FIG. 2, the target nucleic acid measuring apparatus 100 schematically includes a control unit 102, a communication control interface unit 104, an input/output control interface unit 108, and a storage unit 106. The control unit 102 is a CPU and the like that integrally controls the entire operation of the target nucleic acid measuring apparatus 100. The input/output control interface unit 108 is an interface connected to an input unit 112, an output unit 114, and a measuring unit 116. The storage unit 106 is a device that stores various databases, tables or the like. These components of the target nucleic acid measuring apparatus 100 are communicably connected through an arbitrary communication path.

The various databases or files (a measurement data file 106a and a theoretical expression file 106b) stored in the storage unit 106 are storage means such as a fixed disk device. For example, the storage unit 106 stores various programs, tables, files, databases and the like which are used in various processes.

Of these constituent elements of the storage unit 106, the measurement data file 106a stores an amplification amount equivalent of the target nucleic acid measured at each thermal cycle number, corresponding to the thermal cycle number in the nucleic acid amplification reaction (for example, PCR).

The theoretical expression file 106b stores a theoretical expression. The theoretical expression stored in the theoretical expression file 106b includes an environmental coefficient K as an exponent parameter, and a parameter of an initial template amount equivalent N₀. Further, the theoretical expression includes at least one parameter among an amount equivalent to a saturation amount upon the target nucleic acid amplification (hereinafter, referred to as “saturation amount equivalent”) Nₘₐₓ, a reaction acceleration coefficient ρ, and a reaction inhibition coefficient μ.

In the theoretical expression stored in the theoretical expression file 106b, an amplification efficiency of the nucleic acid amplification reaction may be defined by the environmental coefficient K and at least one parameter among the saturation amount equivalent Nₘₐₓ, the reaction acceleration coefficient ρ, and the reaction inhibition coefficient μ. Further, the theoretical expression may express that the amplification amount equivalent Nₙ is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. For example, the theoretical expression may be represented by the following expression.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

The theoretical expression file 106b may store a value of the parameter in the theoretical expression. For example, the theoretical expression file 106b may store, as a fixed value, at least one value of the reaction acceleration coefficient ρ, the reaction inhibition coefficient μ, the environmental coefficient K, and the saturation amount equivalent Nₘₐₓ.

In FIG. 2, the input/output control interface unit 108 controls the input unit 112, the output unit 114, and the measuring unit 116. As the output unit 114, not only a monitor (including a household-use television) but also a speaker may be used (hereinafter, the output unit 114 is sometimes described as a monitor). As the input unit 112, a keyboard, a mouse device, a microphone or the like may be used.

The measuring unit 116 measures an amplification amount equivalent at each thermal cycle number, corresponding to the thermal cycle number in the nucleic acid amplification reaction. As an example, the measuring unit 116 is constituted as a measuring means in a real-time PCR apparatus or the like. In addition, the amplification amount equivalent measured by the measuring unit 116 for each thermal cycle number is stored as measurement data in the measurement data file 106a under the control of the control unit 102.

In FIG. 2, the control unit 102 has an internal memory to store a control program such as an OS (Operating System), a program that defines various procedures, and required data. The control unit 102 performs information processing to execute various processes by these programs or the like. The control unit 102 functionally conceptually includes a theoretical expression fitting unit 102a, and an initial template amount equivalent calculating unit 102b.

The theoretical expression fitting unit 102a fits the theoretical expression stored in the theoretical expression file 106b, to the amplification amount equivalent for each thermal cycle number stored in the measurement data file 106a. That is, the theoretical expression fitting unit 102a determines values of the parameters in the theoretical expression so that the theoretical expression has the best fit to the amplification amount equivalent measured at each thermal cycle number.

Here, the theoretical expression fitting unit 102a may fit the theoretical expression using the least square method. The theoretical expression fitting unit 102a may read out the value of the parameters stored in the theoretical expression file 106b, such as the reaction acceleration coefficient ρ, the reaction inhibition coefficient μ, the environmental coefficient K, or the saturation amount equivalent Nₘₐₓ. And then, the theoretical expression fitting unit 102a may use a predetermined value such as the read-out value of at least one of these parameters as a fixed value when fitting the theoretical expression. The theoretical expression fitting unit 102a may perform the fitting of the theoretical expression by setting the maximum value of the amplification amount equivalents stored in the measurement data file 106a as the saturation amount equivalent Nₘₐₓ. The theoretical expression fitting unit 102a may fix the parameter such as the reaction acceleration coefficient ρ or the reaction inhibition coefficient μ by fitting the theoretical expression to the amplification amount equivalent measured only in a region where the increasing rate Δₙ represented by the following expression is from 0 to 1. That is, the theoretical expression fitting unit 102a may fit the theoretical expression only to the amplification amount equivalent measured at the thermal cycle number of which the increasing rate Δₙ is from 0 to 1. And then, the theoretical expression fitting unit 102a may store, as a fixed value, a value of the parameters such as the reaction acceleration coefficient μ or the reaction inhibition coefficient ρ determined from the fitted theoretical expression, in the theoretical expression file 106b.

$${Δ}_{n} = \frac{{N}_{n}}{{N}_{n - 1}} - 1$$

(Here, Δₙ is the increasing rate; and Nₙ is the amplification amount equivalent at thermal cycle number n.)

The initial template amount equivalent calculating unit 102b calculates the initial template amount equivalent from the theoretical expression fitted by the theoretical expression fitting unit 102a. For example, the initial template amount equivalent calculating unit 102b calculates the initial template amount equivalent N₀ based on the values of the parameters in the theoretical expression, which are the results of the fitting performed by the theoretical expression fitting unit 102a, to output the initial template amount equivalent N₀ to the output unit 114.

An example of the configuration of the target nucleic acid measuring apparatus 100 has been explained hereinbefore. The target nucleic acid measuring apparatus 100 may be communicably connected to a network 300 through a communication device such as a router and a wired or radio communication line such as a leased line. In this case, the communication control interface unit 104 is an interface connected to a communication device (not shown) such as a router connected to the communication line or the like, and performs communication control between the target nucleic acid measuring apparatus 100 and the network 300 (or a communication device such as a router). Namely, the communication control interface unit 104 has a function of performing data communication with another terminal through a communication line. In FIG. 2, the network 300 has a function of connecting the target nucleic acid measuring apparatus 100 and an external system 200 with each other. For example, the Internet is used as the network 300.

The target nucleic acid measuring apparatus 100 may be connected to the external system 200 which provides an external program making a computer serve as the target nucleic acid measuring apparatus, or an external database related to the measurement data or the parameters, through the network 300.

In FIG. 2, the external system 200 is mutually connected to the target nucleic acid measuring apparatus 100 through the network 300. And the external system 200 has a function of providing an external database related to the measurement data, the theoretical expressions, or values of the parameters, and an external program such as a target nucleic acid measuring program that makes an information processing device serve as the target nucleic acid measuring apparatus, to a user. The external system 200 may be designed to serve as a WEB server or an ASP server. The hardware configuration of the external system 200 may be constituted by an information processing device such as a commercially available workstation or personal computer and a peripheral device thereof. The functions of the external system 200 are realized by a CPU, a disk device, a memory device, an input unit, an output unit, a communication control device, and the like in the hardware configuration of the external system 200 and programs which control these devices.

Processing of Target Nucleic Acid Measuring Apparatus 100

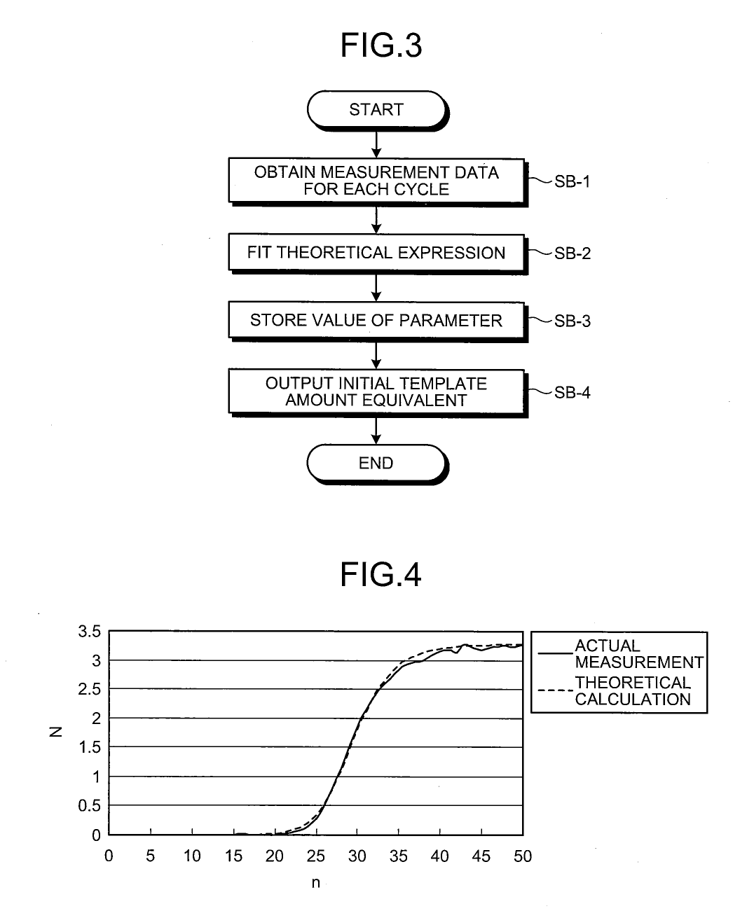

Next, an example of processing of the target nucleic acid measuring apparatus 100 according to Embodiment 1 constructed as described above will be explained below in detail with reference to FIG. 3. FIG. 3 is a flowchart showing an example of the processing of the target nucleic acid measuring apparatus 100 according to Embodiment 1.

The measuring unit 116 of a real-time PCR apparatus or the like measures an amplification amount equivalent at each thermal cycle number, corresponding to the thermal cycle number in the nucleic acid amplification reaction. As shown in FIG. 3, the control unit 102 of the target nucleic acid measuring apparatus 100 obtains measurement data on the amplification amount equivalent measured at each thermal cycle number through the measuring unit 116, and stores the measurement data in the measurement data file 106a (step SB-1).

The theoretical expression fitting unit 102a fits the theoretical expression stored in the theoretical expression file 106b, to the amplification amount equivalent for each thermal cycle number stored in the measurement data file 106a (step SB-2). That is, the theoretical expression fitting unit 102a adjusts and determines values of the parameters in the theoretical expression using the least square method so that the theoretical expression has the best fit to the amplification amount equivalent for each thermal cycle number. In this theoretical expression, an amplification efficiency of the nucleic acid amplification reaction may be defined by not only the environmental coefficient K, but also a parameter such as the saturation amount equivalent Nₘₐₓ, the reaction acceleration coefficient ρ, or the reaction inhibition coefficient As an example, the theoretical expression expresses that the amplification amount equivalent is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. Exemplarily, the theoretical expression is represented by the following expression.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

In this step SB-2, the theoretical expression fitting unit 102a may read out the value of the parameters stored in the theoretical expression file 106b, such as the reaction acceleration coefficient ρ, the reaction inhibition coefficient μ, the environmental coefficient K, or the saturation amount equivalent Nₘₐₓ, and fix any of the parameters. The theoretical expression fitting unit 102a may perform the fitting of the theoretical expression by setting the maximum value of the amplification amount equivalents stored in the measurement data file 106a as the saturation amount equivalent Nₘₐₓ. The theoretical expression fitting unit 102a may evaluate the parameter such as the reaction acceleration coefficient ρ or the reaction inhibition coefficient μ by fitting the theoretical expression to the amplification amount equivalents measured only in a region where the increasing rate Δₙ represented by the following expression is from 0 to 1. And then, the theoretical expression fitting unit 102a may store the evaluated value of the parameter as a fixed value in the theoretical expression file 106b.

$${Δ}_{n} = \frac{{N}_{n}}{{N}_{n - 1}} - 1$$

(Here, Δₙ is the increasing rate; and Nₙ is the amplification amount equivalent at thermal cycle number n.)

The theoretical expression fitting unit 102a stores each value of the parameters of the fitted theoretical expression, in the theoretical expression file 106b (step SB-3). For example, the theoretical expression fitting unit 102a stores, as a result of the fitting, those values of the parameters optimized so that the theoretical expression has the best fit to the amplification amount equivalent using the least square method or the like, in the theoretical expression file 106b.

The initial template amount equivalent calculating unit 102b calculates the initial template amount equivalent N₀ based on the values of the parameters of the theoretical expression, stored in the theoretical expression file 106b, to output the initial template amount equivalent N₀ to the output unit 114 through the input/output control interface unit 108 (step SB-4). An example of the processing of the target nucleic acid measuring apparatus 100 has been described hereinbefore.

According to Embodiment 1, the theoretical expression is fitted to the amplification amount equivalent measured at each thermal cycle number in the nucleic acid amplification reaction, and the initial template amount equivalent N₀ is calculated from the fitted theoretical expression. The theoretical expression includes an environmental coefficient K as an exponent parameter, and a parameter of an initial template amount equivalent N₀, and further includes at least one parameter among a saturation amount equivalent Nₘₐₓ, a reaction acceleration coefficient ρ, and a reaction inhibition coefficient μ. Therefore, according to Embodiment 1, the initial template amount equivalent can be precisely determined using a theoretical expression that allows to be fitted directly to the measured amplification amount equivalent and is capable of precisely reflecting the actual amplification efficiency.

According to Embodiment 1, an amplification efficiency of the nucleic acid amplification reaction is defined in the theoretical expression by the environmental coefficient K and at least one parameter among the saturation amount equivalent Nₘₐₓ, the reaction acceleration coefficient ρ, and the reaction inhibition coefficient μ. Therefore, according to Embodiment 1, the initial template amount equivalent can be more precisely determined using the theoretical expression that is capable of more precisely reflecting the actual amplification efficiency. According to Embodiment 1, the theoretical expression expresses that the amplification amount equivalent is equal to the product of the initial template amount equivalent N₀ and a sequence of the amplification efficiencies to each of which 1 is added for each thermal cycle number. Therefore, according to Embodiment 1, the initial template amount equivalent can be more precisely determined using the theoretical expression that allows to be fitted directly to the measured amplification amount equivalent and is capable of more precisely reflecting the actual amplification efficiency.

According to Embodiment 1, the theoretical expression is represented by the following expression, thereby enabling to determine the initial template amount equivalent more precisely using the theoretical expression that is capable of more precisely reflecting the actual amplification efficiency.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

According to Embodiment 1, the theoretical expression is fitted using the least square method, thereby enabling to perform the fitting and determine the initial template amount equivalent, more precisely by fixing the obtained value of the parameter.

According to Embodiment 1, at least one of the reaction acceleration coefficient ρ, the reaction inhibition coefficient μ, the environmental coefficient K, and the saturation amount equivalent Nₘₐₓ, in the theoretical expression is a fixed value. Therefore, according to Embodiment 1, the initial template amount equivalent can be more precisely determined in a short amount of time.

According to Embodiment 1, any one of the reaction acceleration coefficient ρ and the reaction inhibition coefficient μ or both are calculated to be fixed by fitting the theoretical expression to the amplification amount equivalent measured in a region where an increasing rate represented by the following expression is from 0 to 1. Therefore, according to Embodiment 1, the initial template amount equivalent can be more precisely determined only using an appropriate region of the measured amplification amount equivalents.

$${Δ}_{n} = \frac{{N}_{n}}{{N}_{n - 1}} - 1$$

(Here, Δₙ is the increasing rate; and Nₙ is the amplification amount equivalent at thermal cycle number n.)

According to Embodiment 1, the maximum value of the measured amplification amount equivalents is set as the saturation amount equivalent Nₘₐₓ, thereby enabling to fix the parameter of the saturation amount equivalent Nₘₐₓ using the measurement value and determine the initial template amount equivalent more precisely. The explanation of one example of Embodiment 1 will be ended.

### Example 1

Example 1 according to Embodiment 1 will be described hereinafter with reference with FIGS. 4 to 6.

Theoretical Expression

First, a theoretical expression to be used in Example 1 will be described bellow.

In the real-time PCR system according to this Example 1, the solution needed in the PCR is composed of an appropriate buffer, two sets of complementary oligonucleotide primers, an excess amount of four nucleotide triphosphates, a DNA polymerase, an unknown amount of the target nucleic acid molecules, and the like. For such a composition, the amount of the PCR product, Nₙ, in the PCR reaction is represented by the following expression.

$${N}_{n} = {N}_{n - 1} ( 1 + ρ \frac{{N}_{max} - {N}_{n - 1}}{{N}_{max} + μ {N}_{n - 1}} ) p ( {N}_{zn - 1} , {A}_{zn - 1} , {T}_{x} , {L}_{t} , {L}_{p} ) \qquad ( 1 )$$

(Here, ρ is an amplification coefficient, and is ideally 1, but in practice, the value becomes from 0 to 1 depending on the conditions of the system (experimental conditions) (when ρ=0, no amplification); μ is an inhibition coefficient, and is ideally 0, but under the influence of an amplification inhibitory substance such as pyrophosphoric acid, the value becomes from 0 to ∞ (when μ=ρ, no amplification); and p(N_{zn-1}, A_{zn-1}, Tₓ, Lₜ, Lₚ) is a performance characteristic function for the polymerases, and the value thereof depends on the number of the polymerases at thermal cycle number n-1 in the PCR, N_{zn-1}; specific activity of the polymerase at the thermal cycle number n-1, A_{zn-1}; the extension time, Tₓ (seconds); the base length of the template, Lₜ; and the base length of the primer, Lₚ.)

Since p(N_{zn-1}, A_{zn-1}, Tₓ, Lₜ, Lₚ)=1 when the polymerases function perfectly, the expression (1) becomes the following expression.

$${N}_{n} = {N}_{n - 1} ( 1 + ρ \frac{{N}_{max} - {N}_{n - 1}}{{N}_{max} + μ {N}_{n - 1}} ) \qquad ( 2 )$$

However, under the practical conditions, the polymerase is not believed to be functioning perfectly. Also, since a specific expression of the performance characteristic function for the polymerase, p(N_{zn-1}, A_{zn-1}, Tₓ, Lₜ, Lₚ), is not clearly known, a variable K is introduced in place of this function to change the expression (2) to the following expression in Example 1.

$${N}_{n} = {N}_{n - 1} \{ 1 + {ρ ( \frac{{N}_{max} - {N}_{n - 1}}{{N}_{max} + μ {N}_{n - 1}} )}^{K} \} \qquad ( 3 )$$

Here, Nₘₐₓ in the expressions (1), (2) and (3) denotes a saturation amount of DNA (the amount of DNA saturated when thermal cycles are sufficiently carried out in the PCR), and is represented by the following expression.

Nₘₐₓ=Nₚ₀+N₀ (4)

(Here, Nₚ₀ is the number of the initial primers; and N₀ is the number of the template DNAs.)

According to the expression (3), the number of DNAs at thermal cycle number n in the PCR is represented by the following expression.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \} \qquad ( 5 )$$

Further, according to the expression (3), a DNA increasing rate (amplification efficiency) at thermal cycle number n in the PCR, Δₙ, is represented by the following expression.

$${Δ}_{n} = \frac{{N}_{n}}{{N}_{n - 1}} - 1 = {ρ ( \frac{{N}_{max} - {N}_{n - 1}}{{N}_{max} + μ {N}_{n - 1}} )}^{K} \qquad ( 6 )$$

As is obvious from the expression (6), the relationship between the increasing rate Δₙ and the amplification coefficient ρ is theoretically represented by the following relational expression.

0≦Δₙ≦ρ≦1 (7)

According to this Example 1, the least square fitting is performed based on the theoretical expression shown above (specifically, the expression (5)). Specifically, the coefficients, ρ, μ, K, N₀ and Nₘₐₓ are determined by performing least square fitting of the expression (5) for a plurality of amplification amount equivalents Nₙs that can be detected with high precision. Then, the value of N₀ thus determined is taken as the estimated quantity of the template DNA (initial template amount equivalent). Here, when all of ρ, μ, and Nₘₐₓ are constants (fixed values) common in the conditions of the system, the parameters to be determined by fitting are eliminated, thereby making the calculation easy. Since it is believed that ρ varies according to the conditions of the system, and Nₘₐₓ varies according to the amount of the solution, these parameters may not be fixed values, but variables as they are.

Here, the difference between the theoretical expression of this Example 1 constituted as described above, and conventional theoretical expressions, will be explained.

For example, the expression disclosed in JP-A-8-66199 includes the amounts of the primer and the polymerase as aforementioned. Here, in the expression shown in JP-A-8-66199, when the concentration is re-written by the number of DNAs, and the expression is re-written using an inhibition coefficient, the following expression is obtained.

$${N}_{n} = {N}_{n - 1} {e}_{V} + Min [ \begin{matrix}{N}_{n - 1} {e}_{V} ρ {\{ 1 + \frac{( 1 + μ ) {N}_{n - 1}}{{N}_{p 0} + {N}_{0} - {N}_{n - 1}} \}}^{- 1} , \\ \frac{{N}_{zn - 1} {A}_{zn - 1} {T}_{x}}{{L}_{t} - {L}_{p}}\end{matrix} ] \\ \qquad ( 8 )$$

(Here, Nₙ₋₁ and Nₙ are the numbers of DNA at the thermal cycle numbers n-1 and n in a PCR, respectively; eᵥ is the DNA's probability of survival; ρ is an amplification coefficient; μ is an inhibition coefficient, N₀ is the number of template DNAs; Nₚ₀ is the number of initial primers; N_{zn-1} and A_{zn-1} are the number of polymerases and the specific activity of the polymerase at thermal cycle number n-1 in the PCR; Tₓ is the extension time (seconds); Lₜ is the base length of the template; Lₚ is the base length of the primer; and the saturation amount of DNA, Nₘₐₓ, is given by the following expression.)

Nₘₐₓ=Nₚ₀+N₀ (9)

When the polymerases are fully functioning and eᵥ=1, the following expression is given by the expression (9)

$${N}_{n} = {N}_{n - 1} ( 1 + ρ \frac{{N}_{max} - {N}_{n - 1}}{{N}_{max} + μ {N}_{n - 1}} ) \qquad ( 10 )$$

Therefore, the expression (10) can be represented by the following expression.

$${N}_{n} = {N}_{0} {\prod }_{j = 1}^{n} ( 1 + ρ \frac{{N}_{max} - {N}_{j - 1}}{{N}_{max} + μ {N}_{j - 1}} ) \qquad ( 11 )$$

Next, JP-A-8-66199 discloses that the expression (1) can be represented by the following expression when Nₙ depends on the dysfunction of the polymerases.

$${N}_{n} = {N}_{n - 1} {e}_{V} + \frac{{N}_{zn - 1} {A}_{zn - 1} {T}_{x}}{{L}_{t} - {L}_{p}} \qquad ( 12 )$$

As such, the second term in the expression (12) derived from JP-A-8-66199 does not include Nₙ₋₁, and is not understandable. There is no explanation on the initial number of polymerase, N_{z0}, and the initial specific activity, A_{z0}, and thus the calculation cannot be carried out. Furthermore, the expression (8) employs the smaller of the two argument values in the second term, and the both arguments can be used for fitting in practice. Since it is not known which argument should be employed for the fitting, the expression (8) cannot be utilized in the fitting. Essentially, it is believed that upon performing the fitting, the expression including these two arguments should be arranged into a single expression without arguments, and only when the expression are arranged into one without arguments, a practical system can be established.

Compared with the expression described in this JP-A-8-66199, the theoretical expression (5) used in this Example 1 be utilized in the fitting since this expression is presented as an expression unified throughout the entire thermal cycles of the PCR.

Furthermore, by introducing one exponent parameter (environmental coefficient K) in place of the performance characteristic function for the polymerases which fluctuates with the thermal cycles of the PCR, various parameters can be omitted. Examples of the parameters that can be omitted include the number of polymerases, the specific activity of the polymerase, the extension time, the base length of the template, and the base length of the primer. By omitting these various parameters, fitting of the theoretical expression can be made easy.

In the explanation given above, it was described such that the exponent parameter (environmental coefficient K) is introduced in place of the performance characteristic function for the polymerase. However, parameters to be replaced with this exponent parameter (environmental coefficient K) are not limited to the parameters associated with the polymerase.

That is, in regard to the actual nucleic acid amplification reaction such as PCR and measurements thereof, the actual amplification efficiency based on the measured amplification amount equivalents is influenced not only by the performance characteristics of the polymerase, but also by various known factors or unknown factors. In this Example 1, it was found that by introducing the exponent parameter (environmental coefficient K) into the amplification efficiency in the theoretical expression, those parameters based on such diverse factors can be replaced with the single parameter, and the calculated amplification efficiency can be highly coincident with the actual amplification efficiency.

As an example, those factors already known to exert influence on the environmental coefficient K include the following. That is, examples of the factors associated with the measuring apparatus include the factors resulting from the thermal cycler, such as the error relative to the set temperature, the rate of temperature increase, the rate of temperature decrease or the like as well as the factors resulting from the light measuring unit, such as the sensitivities (of CCD and the light source), the image density conversion treatment or the like. Examples of the factors associated with the application include the heat resistance of the enzymes (polymerase and the like), the difference in the amplification efficiency due to the primer sequence, the difference in the amplification efficiency due to the type of the enzymes (polymerase and the like), and the like. As such, the environmental coefficient K as an exponent parameter that is introduced into the amplification efficiency of the theoretical expression, can be a substitute for these parameters associated with such known factors and unknown factors.

Fitting Method

Next, a specific fitting method according to Example 1 will be explained with reference to FIGS. 4 to 6.

Consideration that should be made when fitting is to reference as much available data as possible. FIG. 4 is a diagram showing measurement data obtained by the measuring apparatus ABI7900 (trade name) manufactured by Applied Biosystems, Inc. In FIG. 4, the solid line represents measurement curve based on the actual measurement values (amplification amount equivalents) of measurement data, and the dashed line represents theoretical curve of the fitted theoretical expression. Thermal cycle number n is shown on the horizontal axis, and an amplification amount equivalent N is shown on the vertical axis.

In FIG. 4, the expression (5) was used as the theoretical expression for the calculation. Under the assumption that the data had been measured until the saturation point was reached, the maximum value (in this case, 3.274499) of the measured amplification amount equivalents was set as the saturation amount equivalent Nₘₐₓ. To facilitate, measurement data until the increasing rate Δₙ reached negative value was recognized as the data up to the saturation points. In the strict sense, since data did not reach the saturation point, iterative calculation for fitting should be further carried out until the finite Nₘₐₓ obtained by simulation is equal to the finite Nₘₐₓ of the raw data (measurement data). The calculation was carried out as K=1 (for example, under the assumption that the polymerase be functioning perfectly).

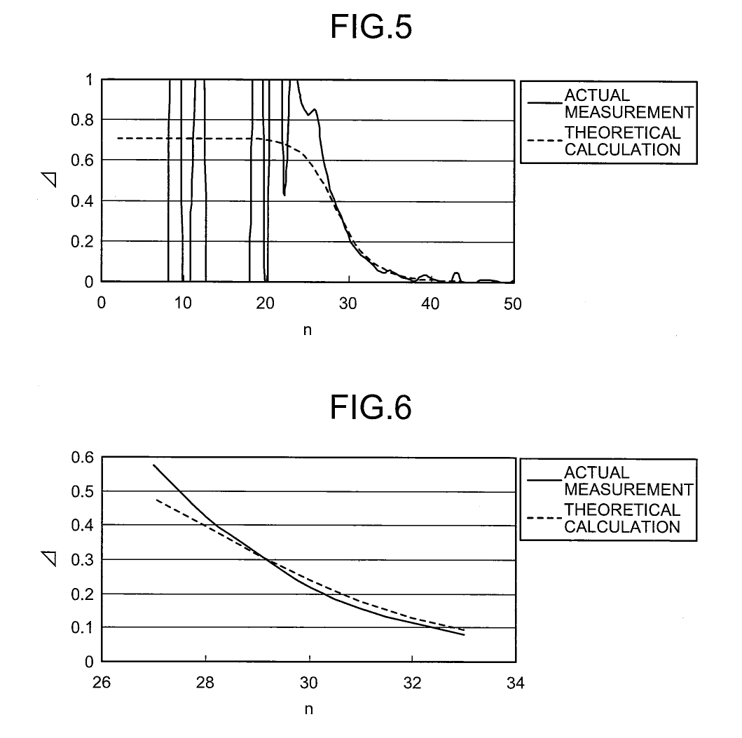

Property of the increasing rate Δₙ based on the expression (6) for this measurement data is depicted in FIG. 5. FIG. 5 is a diagram in which thermal cycle number n is shown on the horizontal axis, and an increasing rate Δₙ is shown on the vertical axis. In FIG. 5, the solid line represents a graph line of the increasing rate Δₙ based on the actual measurement values in the measurement data, and the dashed line represents theoretical curve of the increasing rate Δₙ based on the fitted theoretical expression.

As shown in FIG. 5, the theoretical calculation values satisfy the above relational expression (7) (i.e., 0≦Δₙ≦ρ≦1), however, the actual measurement sometimes does not satisfy this relational expression due to the measurement error (mainly, background noise). Exemplarily, with the exception of data domain where the relational expression (0≦Δₙ≦ρ≦1) is not satisfied, a region to be fitted should be set to other data domain where the increasing rate Δₙ continuously decreases. FIG. 6 is a diagram showing comparison between the actual measurement and the theoretical calculation, pulling out the region to be fitted that satisfies the relational expression “0≦Δₙ≦ρ≦1” from FIG. 5.

As shown in FIG. 6, for this measurement data, there were seven data points in the region to be fitted, where thermal cycle numbers n=27 to 33. Fitting was performed for the amplification amount equivalents Ns at two (n=27 and 33) of the seven data points to evaluate the reaction acceleration coefficient ρ, and the reaction inhibition coefficient μ.

When K=1 in the above expression (6), the following expressions are obtained.

$$\begin{matrix}ρ \frac{{N}_{max} - {N}_{26}}{{N}_{max} + μ {N}_{26}} = {Δ}_{27} & ( 13 ) \\ ρ \frac{{N}_{max} - {N}_{32}}{{N}_{max} + μ {N}_{32}} = {Δ}_{33} & ( 14 )\end{matrix}$$

As shown in the following expressions, ρ and μ are obtained from the expressions (13) and (14).

$$\begin{matrix}ρ = \frac{{N}_{max} {Δ}_{27} {Δ}_{33} ( {N}_{32} - {N}_{26} )}{{Δ}_{33} {Δ}_{32} ( {N}_{max} - {N}_{26} ) - {Δ}_{27} {Δ}_{26} ( {N}_{max} - {N}_{32} )} & ( 15 ) \\ μ = \frac{{N}_{max} \{ {Δ}_{27} ( {N}_{max} - {N}_{32} ) - {Δ}_{33} ( {N}_{max} - {N}_{26} ) \}}{{Δ}_{33} {Δ}_{32} ( {N}_{max} - {N}_{26} ) - {Δ}_{27} {Δ}_{26} ( {N}_{max} - {N}_{32} )} & ( 16 )\end{matrix}$$

By assigning the actual measurement data (the amplification amount equivalents at n=27 and 33) to the expressions (15) and (16), it was evaluated that ρ=1.06 and μ=3.58. Then, when the initial template amount equivalent N₀ was determined so as to correspond with the Δ values between the two points, the N₀ was calculated to be 5×10⁻⁹. According to Example 1, an example performing the two-point fitting (the way to evaluate the coefficients by solving simultaneous equations) was explained to facilitate understanding, however, the present embodiment is not intended to be limited thereto. Multi-point fitting (exemplarily, seven-point fitting) may be performed using the least square method. Since the theoretical expression according to Example 1 was originally made for the purpose of reaction system, the expression does not include detection luminance, but in order to more precisely describe this relationship, a theoretical expression based on the luminance may be used.

### Example 2

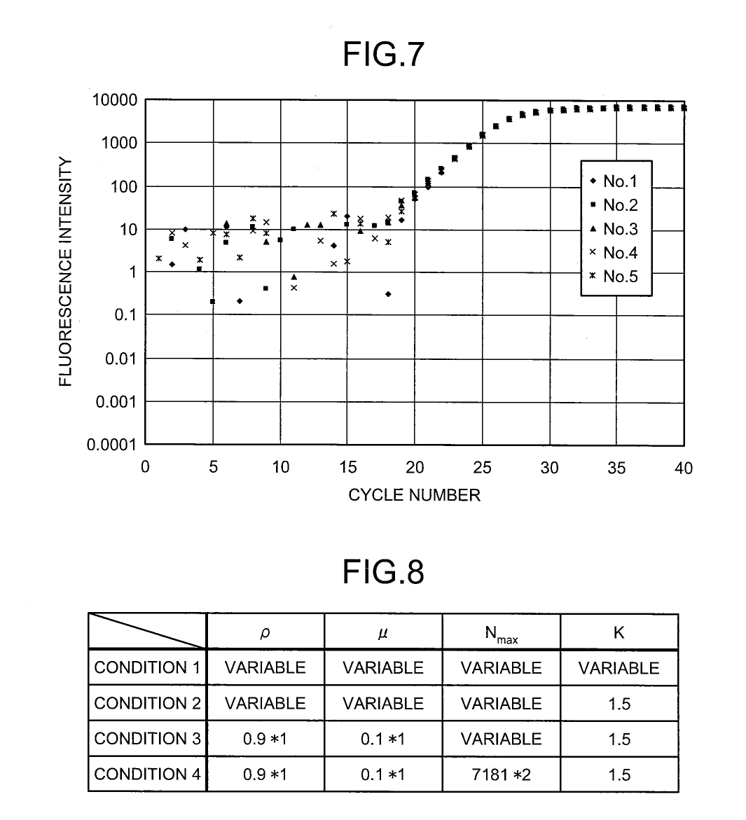

Example 2 according to Embodiment 1 will be described hereinafter with reference with FIGS. 7 to 10.

The inventors of the present application devoted themselves to study to develop a method to be able to determine an initial template amount equivalent using a theoretical expression that allows to be fitted directly to the measured amplification amount equivalent, and is capable of precisely reflecting the actual PCR amplification efficiency. In the result, they developed the following theoretical expression to be fitted to measurement data (fluorescence intensity), that is the measured amplification amount equivalent.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

Here, measurement conditions in Example 2 will be presented below. In this Example 2, a test sample including primers, template DNA, and reagents were prepared as follows for PCR, which is the nucleic acid amplification reaction.

primers for human β-globin: 0.3 μM (final concentration) each

Human Genomic DNA, Male (trade name) (manufactured by Promega Corporation, Cat# G1471) as the template DNA: 500 pg/μl (final concentration)

2× Brilliant II SYBR Green qPCR master mix (trade name) (manufactured by Strategene, Inc.) as the reagents: 1× dilution concentration (final concentration)

PCR was performed under the following conditions, and measurement data of cycle number versus fluorescence intensity was obtained. That is, the amplification amount equivalent is represented by the fluorescence intensity in Example 2.

real-time PCR apparatus: ABI Prism 7900HT (trade name) (manufactured by Applied Biosystems, Inc.)

PCR cycle condition: 95° C./10 minutes→(95° C./30 seconds→60° C./1 minute)×40 cycles

test sample: 30 μl each of identical test samples No. 1 to No. 5 were prepared by the above method.

The PCR result obtained by the above measurement conditions will be presented below. FIG. 7 is a diagram showing the measurement result of the amplification curve, which is the result of cycle number versus fluorescence intensity in each of the test samples.

As shown in FIG. 7, when the cycle number was less than 18, fluorescence intensity (that is, amplification amount equivalent) could not be obtained with accuracy since the fluorescence intensity was weaker than fluorescence detection sensitivity. Therefore, the initial template amount equivalent could not be evaluated only from the measurement data shown in FIG. 7.

According to Example 2, the initial template amount equivalent, N₀, was calculated for each of the test samples by fitting the following theoretical expression developed by the inventors of the present application to the amplification curve as the measurement result.

$$Theoretical Expression \text{:} Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

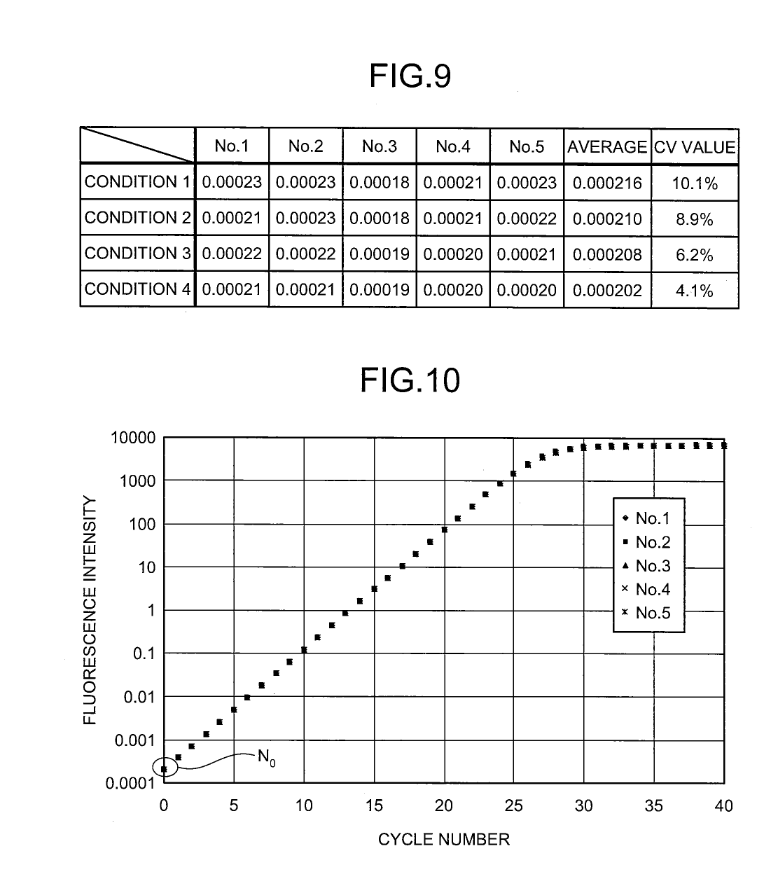

Here, FIG. 8 is a diagram showing conditions of the theoretical expression. FIG. 9 is a diagram showing the calculation result of the template amount equivalent N₀ according to Example 2. In FIG. 8, “*1” represents that values of ρ and μ for No. 1 to No. 5 were calculated from the measurement result at cycles 26 to 34 where an increasing rate Δₙ represented by the following expression is from 0 to 1, and mean values thereof were fixed in the theoretical expression. “*2” represents that the maximum value of fluorescence intensity for each of No. 1 to No. 5 was fitted in the theoretical expression.

$$Increasing Rate \text{:} {Δ}_{n} = \frac{{N}_{n}}{{N}_{n - 1}} - 1$$

(Here, Nₙ denotes the amplification amount equivalent at thermal cycle number n.)

As shown in FIG. 9, a value of N₀ could be calculated for each of the conditions. The result revealed that there is little variability between the test samples for each of the conditions shown in FIG. 8, and there is little variability between the conditions.

FIG. 10 is a diagram showing the theoretical calculation values for the test samples No. 1 to No. 5, obtained on the condition 4. As shown in FIG. 10, it was confirmed that use of the above theoretical expression enables to calculate the initial template amount equivalent N₀ that could not be calculated only from measurement result. It is not shown in the figures, but was confirmed that the initial template amount equivalent N₀ could be calculated for all the conditions Nos. 1, 2 and 3 as well as No. 4.

As described above, according to Example 2, the theoretical expression is fitted directly to the fluorescence intensity, which is the measurement result, without conversion to nucleic acid concentration or the like, thereby enabling to precisely calculate a value equivalent to the initial template amount (initial template amount equivalent).

That is, according to Example 2, the theoretical expression can be fitted to fluorescence intensity as the amplification amount equivalent without conversion to molar concentration of dsDNA, and fluorescence intensity at cycle number 0, which is the initial template amount equivalent in the amplification reaction mixture, can be calculated precisely. The result reflects the actual amplification efficiency of PCR with high accuracy without using standard curve, thereby promoting accuracy of the obtained initial template amount, and enabling to process large number of amplification reaction mixtures whose concentrations are not known. The explanation of Example 2 will be ended.

### Example 3

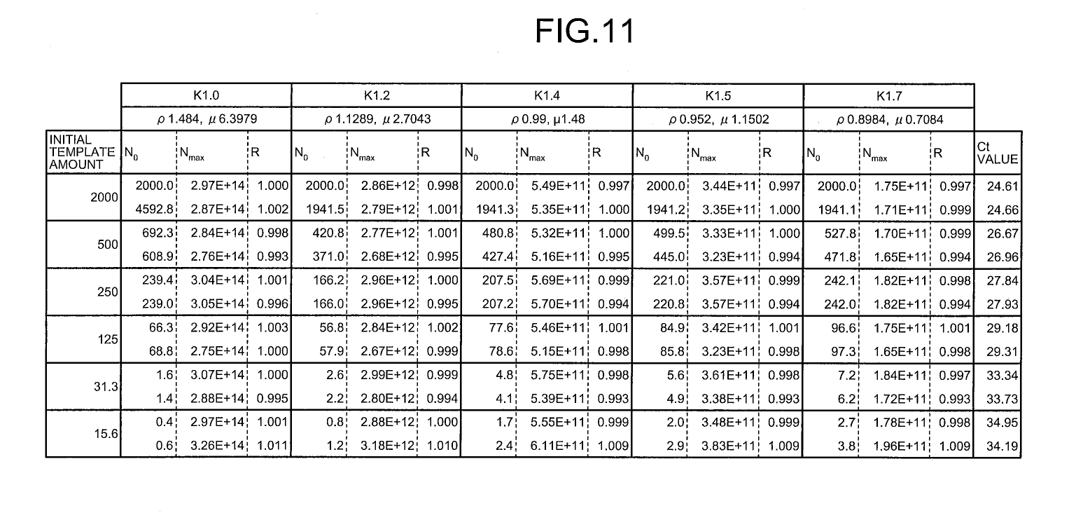

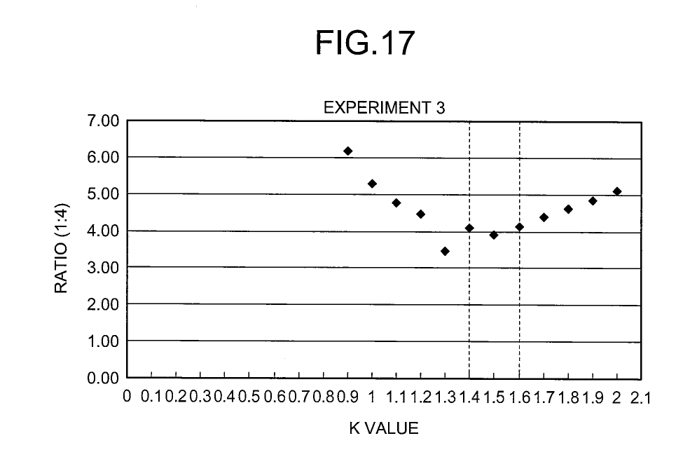

Example 3 according to Embodiment 1 will be described hereinafter with reference with FIGS. 11 to 17. In this Example 3, scopes of values of the parameters in the following theoretical expression were examined.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

FIGS. 11 to 14 are diagrams showing results of fitting the theoretical expression to measurement data using values of ρ and μ obtained by fitting the theoretical expression for standard sample having a known template amount after determining a value of the environmental coefficient K within the scope of K=1.0 to 1.7. That is, a ratio between the initial template equivalents N₀ of two samples were obtained using ρ and μ calculated by fitting the theoretical expression for one standard sample with fixed K. The measurement data shown in FIGS. 11 to 14 were separate experiments, “071010”, “071108”, “080116” and “080828”, conducted on different days, respectively. In each of the figures, No depicts a value of the initial template amount equivalent calculated based on the ratio between the initial template equivalents N₀ of two samples, Nₘₐₓ is a value of calculated Nₘₐₓ, and R is a correlation coefficient of the expressions.

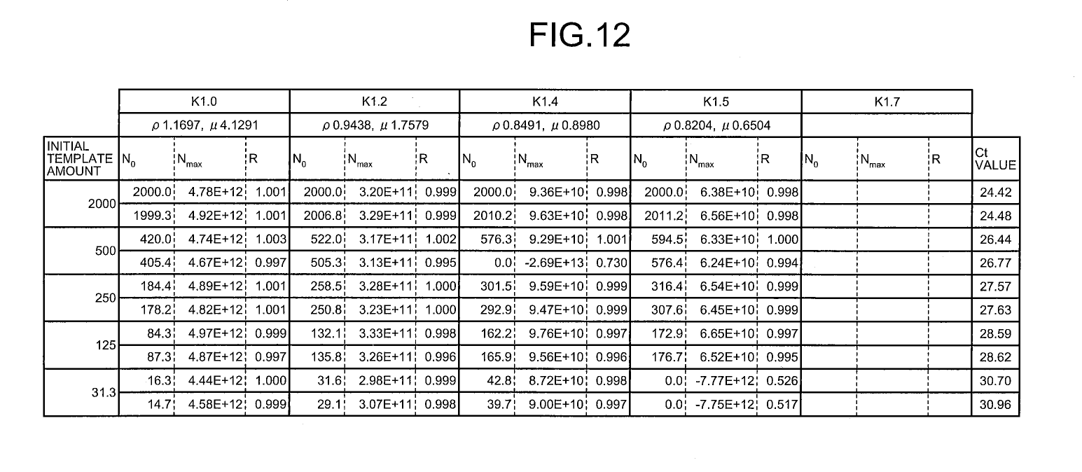

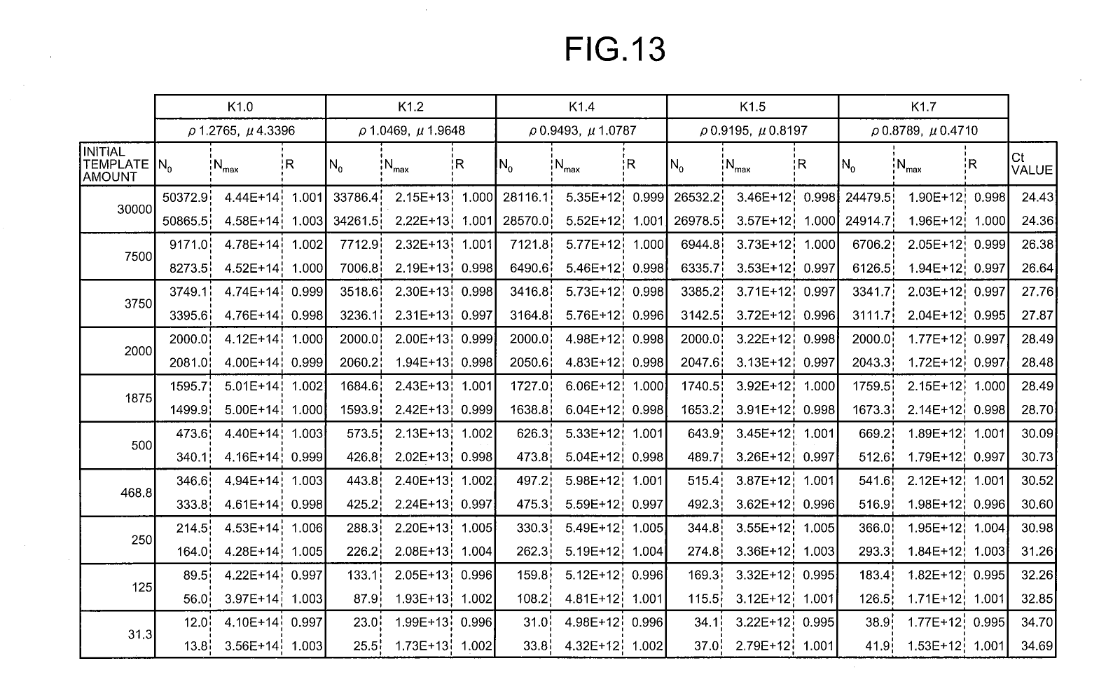

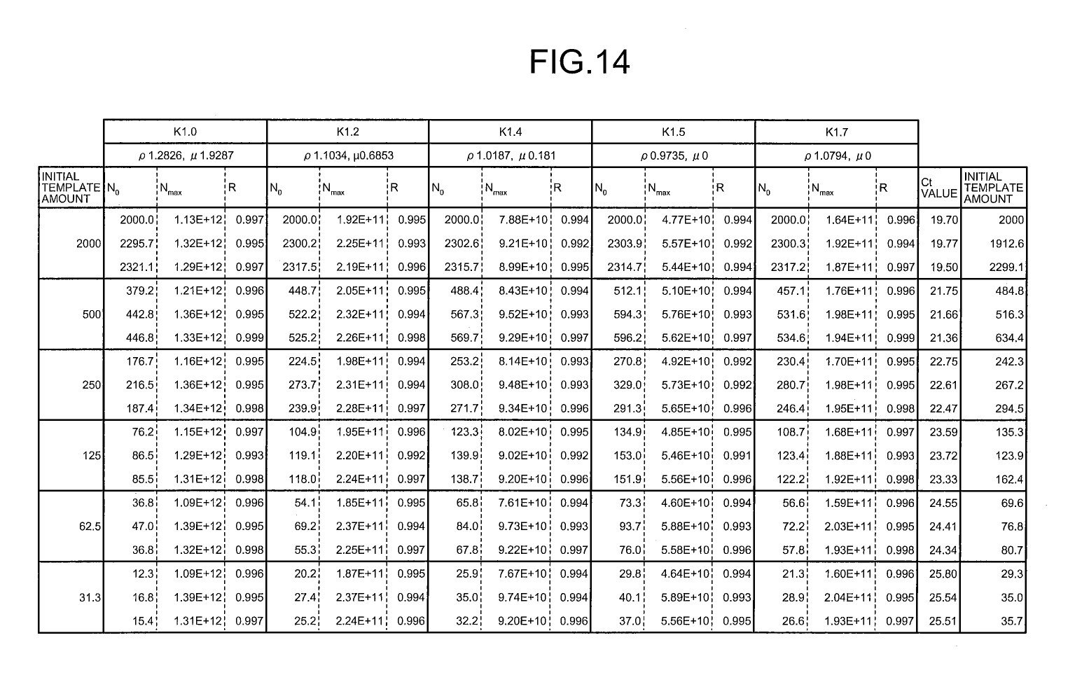

In the result, when comparing the prepared known initial template amount with the calculated initial template amount for each of the measurement data of FIGS. 11 to 14, they are well coincident with each other to be optimized when K=1.5 for the measurement data of FIG. 11. For the rest, the initial template amounts could be optimally calculated with maximum accuracy when K=1.2 for the measurement data of FIG. 12, when K=1.2 for the measurement data of FIG. 13, and when K=1.4 for the measurement data of FIG. 14.

These results revealed that according to Example 3, the initial template amount can be more precisely calculated, compared to the conventional example (that is, K=1.0), in which an environmental coefficient K as an exponential parameter is not introduced into the amplification efficiency in the theoretical expression. As shown in the rightmost column of FIG. 14, the initial template amount was calculated from Ct value in consideration of the amplification efficiency by standard curve method, for comparison with the conventional example using Ct value. As such, according to Example 3, a larger number of test samples can be processed because there is no need to create a standard curve, and the initial template amount can be calculated with similar or higher accuracy compared to the conventional example using Ct value.

Considering scopes of values of the parameters in the theoretical expression, the reaction acceleration coefficient ρ is ideally 0<ρ<1 (see the expression (7) of Example 1), but is not limited thereto according to applications or apparatuses. The inhibition coefficient μ is ideally from 0 to ∞ (see Example 1), and the environmental coefficient K is proved to be approximately from 1 to 2 in practice (here, a result of further studying a value of K will be shown).

To further study possible scope of a value of the parameter of the environmental coefficient K, the following Experiments 1 to 3 were conducted. In this method, two samples having known template amounts (ratio of the amount is 1:4) were prepared, the theoretical expression was fitted for one of the standard sample with fixed K, and a ratio between the initial template amount equivalents N₀ of the two samples using the ρ and μ calculated by the fitting. K was fixed in increments of 0.1 within the range of 0 to 2.0. The following Experiments 1 to 3 are independent experiments from each another.

Here, an experimental condition of Experiments 1 and 2 will be shown (in Experiments 1 and 2, the experimental conditions are identical, but they conducted on different days). The composition of PCR reaction mixture (final concentration (f.c.)) was prepared as follows:

2× Universal PCR Master Mix (trade name) (manufactured by Applied Biosystems, Inc.): 1× dilution concentration primers for human β-globin: 0.3 μM each

TaqMan Probe (trade name): 0.2 μM

Human Genomic DNA: 2000 pg/μl, 500 pg/μl (manufactured by Promega Corporation, Cat# G1471)

Total: 20 μl

As a PCR condition of Experiments 1 and 2, a real-time PCR apparatus, ABI Prism 7900HT (trade name) (manufactured by Applied Biosystems, Inc.) was used. A PCR cycle condition of Experiments 1 and 2 was follows: 95° C./10 minutes→(95° C./15 seconds→60° C./1 minute)×40 cycles.

The experimental conditions of Experiment 3 will be presented below. The composition of PCR reaction mixture (f.c.) was prepared as follows:

2× Brilliant II SYBR Green qPCR master mix (trade name): 1× dilution concentration (manufactured by Strategene, Inc.)

primers for human β-globin: 0.34M each

Human Genomic DNA (trade name): 2000 pg/μl, 500 pg/μl (manufactured by Promega Corporation, Cat#G1471)

Total: 20 μl

As a PCR condition of Experiment 3, a real-time PCR apparatus, ABI Prism 7900HT (trade name) (manufactured by Applied Biosystems, Inc.): was used. A PCR cycle condition of Experiment 3 was follows: 95° C./10 minutes→(95° C./30 seconds→60° C./1 minute)×40 cycles.

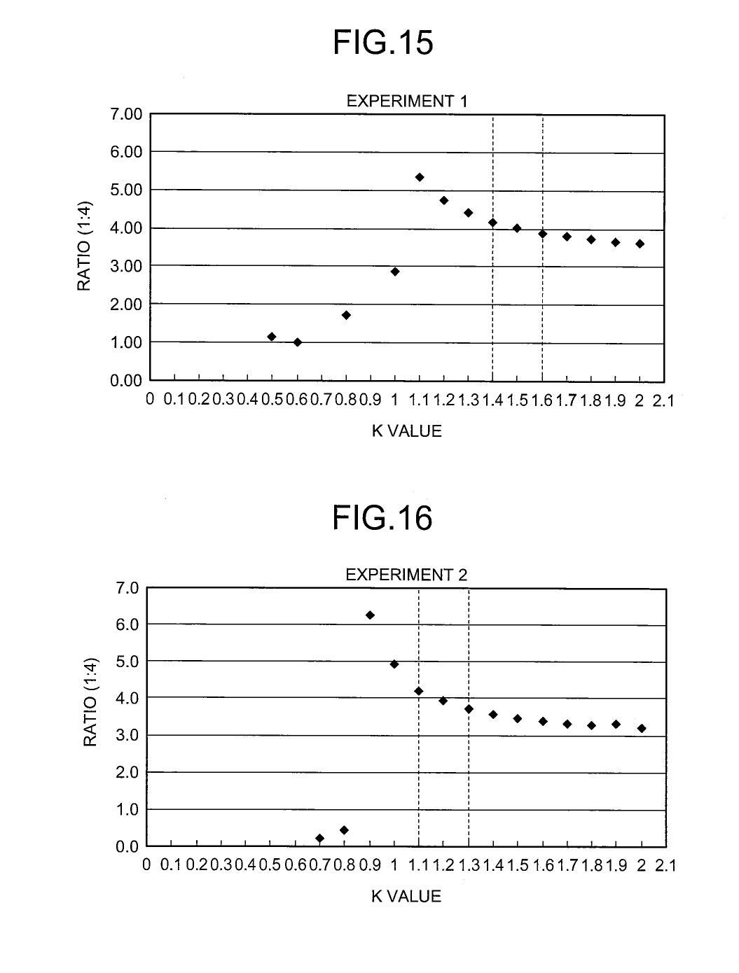

FIGS. 15 to 17 are diagrams showing a ratio of the initial template amount equivalent N₀ calculated using the measurement data of Experiments 1 to 3 in each of which 2000 pg/μl and 500 pg/μl of the initial template amounts were prepared. In each of the figures, a region between the dashed lines represents scope of K value where favorable results were obtained, that is, the calculated ratio is approximately 4, compared with the prepared ratio of the initial amounts (1:4). As such, it is optimized when K=1.4 to 1.6 in Experiment 1, when K=1.1 to 1.3 in Experiment 2, and when K=1.4 to 1.6 in Experiment 3. Thus, the explanation of Example 3 will be ended.

### Embodiment 2

### Overview of Embodiment 2

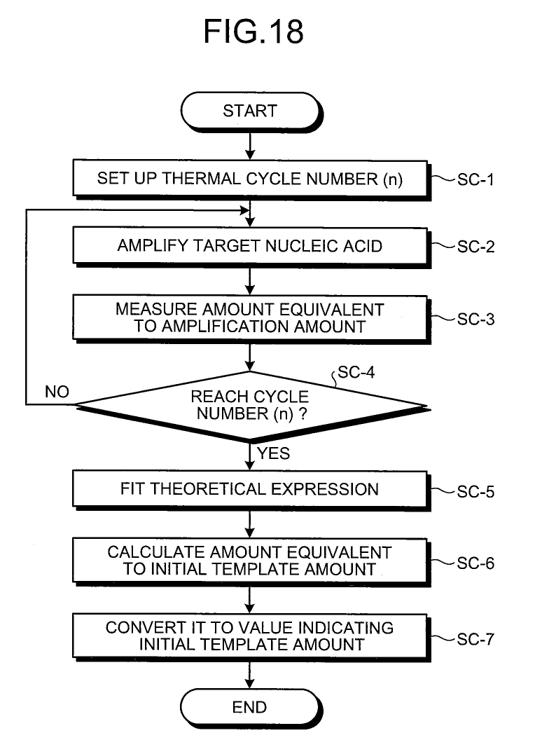

Hereinafter, an overview of Embodiment 2 will be explained with reference to FIG. 18, and then a configuration and processing of Embodiment 2 will be explained in detail. FIG. 18 is a flowchart for explaining an overview of Embodiment 2.

As shown in FIG. 18, according to Embodiment 2, first, the thermal cycle number (n) of a nucleic acid amplification reaction is set up (step SC-1). That is, the thermal cycle number (n) is set up for repeatedly performing the nucleic acid amplification reaction.

According to Embodiment 2, the nucleic acid amplification reaction of the test sample is performed to amplify the target nucleic acid (step SC-2).

According to Embodiment 2, an amount equivalent to an amplification amount of the target nucleic acid (hereinafter, referred to as “amplification amount equivalent”) is measured corresponding to the thermal cycle number (step SC-3). Here, the “amplification amount” means the total amount of the target nucleic acid amplified from thermal cycle number 1 to the thermal cycle number when measuring. Also, the “amplification amount equivalent” is an amount equivalent to the amplification amount of the target nucleic acid, obtained by an arbitrary label. For example, the “amplification amount equivalent” is a signal intensity detected from an intercalator that emits the signal in the presence of a double-strand DNA, or a signal intensity detected from a reporter molecule that emits the signal according to an extension reaction during the amplification reaction.

According to Embodiment 2, it is determined whether current thermal cycle number has reached the set thermal cycle number (n) (step SC-4), and when the current thermal cycle number has not reached the set thermal cycle number (n) (step SC-4, No), the process is returned to the step SC-2. On the other hand, when the current thermal cycle number has reached the set thermal cycle number (n) (step SC-4, Yes), the nucleic acid amplification reaction and the measurement of the amplification amount equivalent according the reaction are finished.

According to Embodiment 2, a theoretical expression is fitted to the amplification amount equivalent measured in each thermal cycle (step SC-5). The theoretical expression according to Embodiment 2 includes an environmental coefficient as an exponent parameter, and a parameter of an amount equivalent to an initial template amount in the test sample (hereinafter, referred to as “initial template amount equivalent”). Further, this theoretical expression includes at least one parameter among an amount equivalent to a saturation amount upon the target nucleic acid amplification, a reaction acceleration coefficient, and a reaction inhibition coefficient. That is, according to Embodiment 2, the parameters of the theoretical expression are determined in this step SC-5 so as to fit the theoretical expression to the amplification amount equivalent measured in each thermal cycle.

In the theoretical expression, an amplification efficiency of the nucleic acid amplification reaction may be defined by the environmental coefficient and at least one parameter among the amount equivalent to the saturation amount upon the target nucleic acid amplification, the reaction acceleration coefficient, and the reaction inhibition coefficient. The theoretical expression may express that the amplification amount equivalent is equal to the product of the initial template amount equivalent and a sequence of the amplification efficiencies to each of which 1 is added for each thermal cycle number. According to Embodiment 2, the theoretical expression may be fitted using the least square method, or at least one of the parameters may be a fixed value.

According to Embodiment 2, the initial template amount equivalent is calculated from the theoretical expression fitted to the amplification amount equivalent (step SC-6). For example, according to Embodiment 2, the initial template amount equivalent is calculated based on the theoretical expression in which the parameters have been determined by fitting.

According to Embodiment 2, the calculated initial template amount equivalent is converted to the value indicating the initial template amount (copy number, mass, amount of substance, concentration, or the like) (step SC-7). An example using a measurement value of a signal from an intercalator that is added to the test sample, and emits the signal only in the presence of a double-strand DNA but not in the absence of the double-strand DNA, will be explained. In this example, the initial template amount equivalent is converted to the value indicating the initial template amount at the following steps a) to d).

step a): a nucleic acid amplification reaction of a standard sample having the same target nucleic acid as the test sample is performed under the same condition as the test sample, an amplification amount of the amplification product of the target nucleic acid is measured, and a dilution series of the amplification product to which the intercalator is added under the same conditions as the test sample, is prepared.

step b): the signal from the intercalator in the dilution series prepared at the step a) is measured under the same conditions as the test sample to obtain the measurement value.

step c): the relational expression between the measurement value obtained at the step b) and the concentration of the dilution series prepared at the step a) are determined.

step d): the initial template amount equivalent is converted to the value indicating the initial template amount based on the relational expression determined at the step c).

The above converting method is one example, and the initial template amount equivalent may be converted to the value indicating the initial template amount by similar way of the above steps a) to d) using a reporter molecule that emits a signal according to an extension reaction during the nucleic acid amplification reaction, in place of the intercalator. When using the reporter molecule, a dilution series of a fluorescent molecule that emits the same signal as the reporter molecule, may be prepared at the step a).

An overview of Embodiment 2 has been explained hereinbefore. According to Embodiment 2, a value indicating the initial template amount equivalent can be precisely determined using a theoretical expression that allows to be fitted directly to the measured amplification amount equivalent and is capable of precisely reflecting the actual amplification efficiency.

Configuration of Target Nucleic Acid Measuring Apparatus

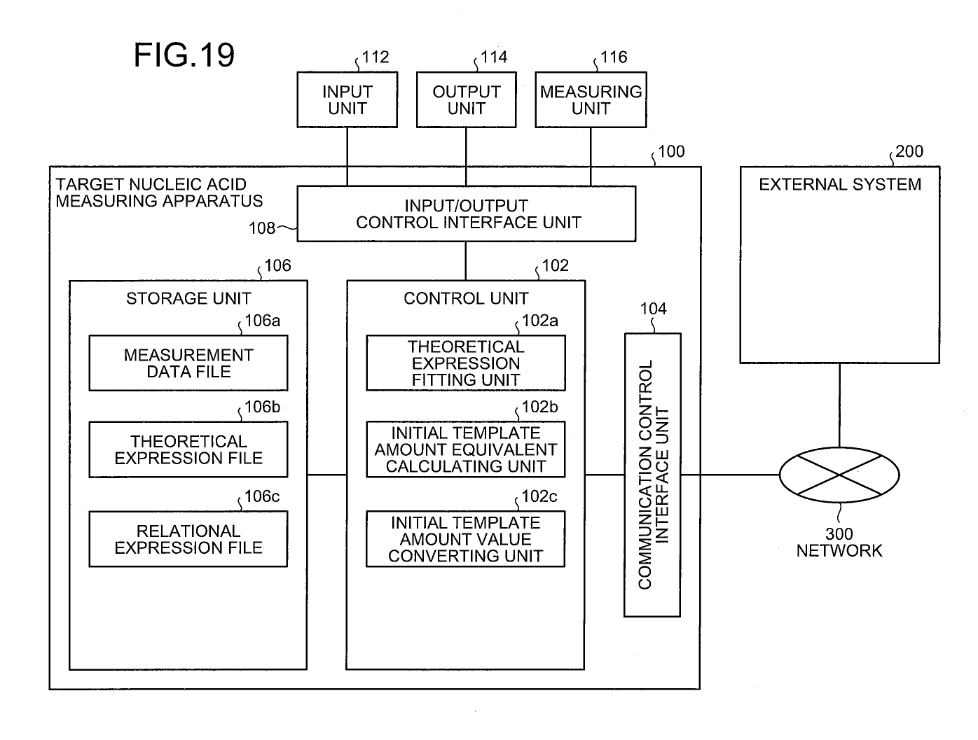

Next, a configuration of a target nucleic acid measuring apparatus according to Embodiment 2 will be explained below with reference to FIG. 19. FIG. 19 is a block diagram showing an example of a configuration of a target nucleic acid measuring apparatus 100 according to Embodiment 2 to which the present invention is applied, and schematically depicts only a part related to Embodiment 2 in the configuration.

In FIG. 19, the target nucleic acid measuring apparatus 100 schematically includes a control unit 102, a communication control interface unit 104, an input/output control interface unit 108, and a storage unit 106. The control unit 102 is a CPU and the like that integrally controls the entire operation of the target nucleic acid measuring apparatus 100. The input/output control interface unit 108 is an interface connected to an input unit 112, an output unit 114, and a measuring unit 116. The storage unit 106 is a device that stores various databases, tables or the like. These components of the target nucleic acid measuring apparatus 100 are communicably connected through an arbitrary communication path.

The various databases or files (a measurement data file 106a, a theoretical expression file 106b, and a relational expression file 106c) stored in the storage unit 106 are storage means such as a fixed disk device. For example, the storage unit 106 stores various programs, tables, files, databases and the like which are used in various processes.

Of these constituent. elements of the storage unit 106, the measurement data file 106a stores an amplification amount equivalent of the target nucleic acid measured at each thermal cycle number, corresponding to the thermal cycle number in the nucleic acid amplification reaction (for example, PCR).

The theoretical expression file 106b stores a theoretical expression. The theoretical expression stored in the theoretical expression file 106b includes an environmental coefficient K as an exponent parameter, and a parameter of an initial template amount equivalent N₀. Further, the theoretical expression includes at least one parameter among an amount equivalent to a saturation amount upon the target nucleic acid amplification (hereinafter, referred to as “saturation amount equivalent”) Nₘₐₓ, a reaction acceleration coefficient ρ, and a reaction inhibition coefficient μ.

In the theoretical expression stored in the theoretical expression file 106b, an amplification efficiency of the nucleic acid amplification reaction may be defined by the environmental coefficient K and at least one parameter among the saturation amount equivalent Nₘₐₓ, the reaction acceleration coefficient ρ, and the reaction inhibition coefficient μ. Further, the theoretical expression may express that the amplification amount equivalent Nₙ is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. For example, the theoretical expression may be represented by the following expression.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

The theoretical expression file 106b may store a value of the parameter in the theoretical expression. For example, the theoretical expression file 106b may store, as a fixed value, at least one value of the reaction acceleration coefficient ρ, the reaction inhibition coefficient μ, the environmental coefficient K, and the saturation amount equivalent Nₘₐₓ.

The relational expression file 106c stores a relational expression for converting the initial template amount equivalent to a value indicating the initial template amount. That is, the relational expression stored in the relational expression file 106c is a relational expression for conversion of a unit of the initial template amount equivalent and the amplification amount equivalent into a unit of the initial template amount and the amplification amount of the target nucleic acid. For example, this relational expression is a relational expression between a measurement value used for representing the amplification amount equivalent (for example, fluorescence intensity or signal measurement value) and a value indicating an amount (template amount) of the target nucleic acid (for example, copy number, mass, amount of substance, or concentration).

In FIG. 19, the input/output control interface unit 108 controls the input unit 112, the output unit 114, and the measuring unit 116. As the output unit 114, not only a monitor (including a household-use television) but also a speaker may be used (hereinafter, the output unit 114 is sometimes described as a monitor). As the input unit 112, a keyboard, a mouse device, a microphone or the like may be used.

The measuring unit 116 measures an amplification amount equivalent at each thermal cycle number, corresponding to the thermal cycle number in the nucleic acid amplification reaction. As an example, the measuring unit 116 is constituted as a measuring means in a real-time PCR apparatus or the like. In addition, the amplification amount equivalent measured by the measuring unit 116 for each thermal cycle number is stored as measurement data in the measurement data file 106a under the control of the control unit 102.

In FIG. 19, the control unit 102 has an internal memory to store a control program such as an OS (Operating System), a program that defines various procedures, and required data. The control unit 102 performs information processing to execute various processes by these programs or the like. The control unit 102 functionally conceptually includes a theoretical expression fitting unit 102a, an initial template amount equivalent calculating unit 102b, and an initial template amount value converting unit 102c.

The theoretical expression fitting unit 102a fits the theoretical expression stored in the theoretical expression file 106b, to the amplification amount equivalent for each thermal cycle number stored in the measurement data file 106a. That is, the theoretical expression fitting unit 102a determines values of the parameters in the theoretical expression so that the theoretical expression has the best fit to the amplification amount equivalent measured at each thermal cycle number.

Here, the theoretical expression fitting unit 102a may fit the theoretical expression using the least square method. The theoretical expression fitting unit 102a may read out the value of the parameters stored in the theoretical expression file 106b, such as the reaction acceleration coefficient ρ, the reaction inhibition coefficient μ, the environmental coefficient K, or the saturation amount equivalent Nₘₐₓ. And then, the theoretical expression fitting unit 102a may use a predetermined value such as the read-out value of at least one of these parameters as a fixed value when fitting the theoretical expression. The theoretical expression fitting unit 102a may perform the fitting of the theoretical expression by setting the maximum value of the amplification amount equivalents stored in the measurement data file 106a as the saturation amount equivalent Nₘₐₓ. The theoretical expression fitting unit 102a may fix the parameter such as the reaction acceleration coefficient ρ or the reaction inhibition coefficient μ by fitting the theoretical expression to the amplification amount equivalent measured only in a region where the increasing rate Δₙ represented by the following expression is from 0 to 1. That is, the theoretical expression fitting unit 102a may fit the theoretical expression only to the amplification amount equivalent measured at the thermal cycle number of which the increasing rate Δₙ is from 0 to 1. And then, the theoretical expression fitting unit 102a may store, as a fixed value, the value of the parameter such as the reaction acceleration coefficient ρ or the reaction inhibition coefficient μ determined from the fitted theoretical expression, in the theoretical expression file 106b.

$${Δ}_{n} = \frac{{N}_{n}}{{N}_{n - 1}} - 1$$

(Here, Δₙ is the increasing rate; and Nₙ is the amplification amount equivalent at thermal cycle number n.)

The initial template amount equivalent calculating unit 102b calculates the initial template amount equivalent from the theoretical expression fitted by the theoretical expression fitting unit 102a. For example, the initial template amount equivalent calculating unit 102b calculates the initial template amount equivalent N₀ based on the values of the parameters in the theoretical expression, which are the results of the fitting performed by the theoretical expression fitting unit 102a, to output the initial template amount equivalent N₀ to the output unit 114.

The initial template amount value converting unit 102c converts the initial template amount equivalent calculated by the initial template amount equivalent calculating unit 102b to a value indicating the initial template amount based on the relational expression stored in the relational expression file 106c. Examples of the unit of the initial template amount include the copy number (copy), mass (pg, ng), amount of substance (mol), concentration (mol/ml), and the like.

The initial template amount value converting unit 102c may determine a relational expression between a measurement value measured through the measuring unit 116 under the same condition as the test sample with respect to a dilution series prepared by the following preparative method (I), (II) or (III), and the concentration of the dilution series input through the input unit 112. The determined relational expression may be stored in the relational expression file 106c.

Preparative Method (I): a nucleic acid amplification reaction of a standard sample having the same target nucleic acid as the test sample is performed under the same condition as the test sample, an amplification amount (concentration or the like) of the amplification product of the target nucleic acid is measured, and a dilution series of the amplification product to which the intercalator is added is prepared under the same conditions as the test sample.

Preparative Method (II): a dilution series of a fluorescent molecule that emits the same signal as the reporter molecule that is added to the test sample is prepared.

Preparative Method (III): a nucleic acid amplification reaction of a standard sample having the same target nucleic acid as the test sample and having the added reporter molecule, is performed under the same condition as the test sample, an amplification amount (concentration or the like) of the amplification product of the target nucleic acid is measured, and a dilution series of the amplification product is prepared under the same conditions as the test sample.

An example of the configuration of the target nucleic acid measuring apparatus 100 has been explained hereinbefore. The target nucleic acid measuring apparatus 100 may be communicably connected to a network 300 through a communication device such as a router and a wired or radio communication line such as a leased line. In this case, the communication control interface unit 104 is an interface connected to a communication device (not shown) such as a router connected to the communication line or the like, and performs communication control between the target nucleic acid measuring apparatus 100 and the network 300 (or a communication device such as a router). Namely, the communication control interface unit 104 has a function of performing data communication with another terminal through a communication line. In FIG. 19, the network 300 has a function of connecting the target nucleic acid measuring apparatus 100 and an external system 200 with each other. For example, the Internet is used as the network 300.

The target nucleic acid measuring apparatus 100 may be connected to the external system 200 which provides an external program making a computer serve as the target nucleic acid measuring apparatus, or an external database related to the measurement data or the parameters, through the network 300.

In FIG. 19, the external system 200 is mutually connected to the target nucleic acid measuring apparatus 100 through the network 300. And the external system 200 has a function of providing an external database related to the measurement data, the theoretical expressions, or values of the parameters, and an external program such as a target nucleic acid measuring program that makes an information processing device serve as the target nucleic acid measuring apparatus, to a user. The external system 200 may be designed to serve as a WEB server or an ASP server. The hardware configuration of the external system 200 may be constituted by an information processing device such as a commercially available workstation or personal computer and a peripheral device thereof. The functions of the external system 200 are realized by a CPU, a disk device, a memory device, an input unit, an output unit, a communication control device, and the like in the hardware configuration of the external system 200 and programs which control these devices.

Processing of Target Nucleic Acid Measuring Apparatus 100

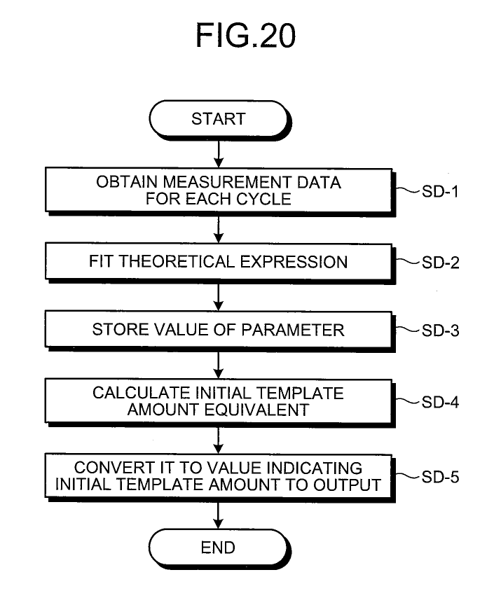

Next, an example of processing of the target nucleic acid measuring apparatus 100 according to Embodiment 2 constructed as described above will be explained below in detail with reference to FIG. 20. FIG. 20 is a flowchart showing an example of the processing of the target nucleic acid measuring apparatus 100 according to Embodiment 2.

The measuring unit 116 of a real-time PCR measuring apparatus or the like measures an amplification amount equivalent (for example, a measurement value of a signal from the intercalator or the reporter molecule) at each thermal cycle number, corresponding to the thermal cycle number in the nucleic acid amplification reaction. As shown in FIG. 20, the control unit 102 of the target nucleic acid measuring apparatus 100 obtains measurement data on the amplification amount equivalent measured at each thermal cycle number through the measuring unit 116, and stores the measurement data in the measurement data file 106a (step SD-1).

The theoretical expression fitting unit 102a fits the theoretical expression stored in the theoretical expression file 106b, to the amplification amount equivalent for each thermal cycle number stored in the measurement data file 106a (step SD-2). That is, the theoretical expression fitting unit 102a adjusts and determines values of the parameters in the theoretical expression using the least square method so that the theoretical expression has the best fit to the amplification amount equivalent for each thermal cycle number. In this theoretical expression, an amplification efficiency of the nucleic acid amplification reaction may be defined by not only the environmental coefficient K, but also a parameter such as the saturation amount equivalent Nₘₐₓ, the reaction acceleration coefficient ρ, or the reaction inhibition coefficient μ. As an example, the theoretical expression expresses that the amplification amount equivalent is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. Exemplarily, the theoretical expression is represented by the following expression.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

The theoretical expression fitting unit 102a stores each value of the parameters of the fitted theoretical expression, in the theoretical expression file 106b (step SD-3). For example, the theoretical expression fitting unit 102a stores, as a result of the fitting, those values of the parameters optimized so that the theoretical expression has the best fit to the amplification amount equivalent using the least square method or the like, in the theoretical expression file 106b.

The initial template amount equivalent calculating unit 102b calculates the initial template amount equivalent N₀ based on the values of the parameters of the theoretical expression, stored in the theoretical expression file 106b, to output the initial template amount equivalent N₀ to the output unit 114 through the input/output control interface unit 108 (step SD-4).

The initial template amount value converting unit 102c converts the initial template amount equivalent N₀ calculated by the initial template amount equivalent calculating unit 102b to a value indicating the initial template amount (concentration or the like) based on the relational expression stored in the relational expression file 106c (step SD-5). The initial template amount value converting unit 102c may determine a relational expression between a measurement value measured through the measuring unit 116 under the same condition as the test sample with respect to a dilution series prepared by the following preparative method (I), (II) or (III), and the concentration of the dilution series input through the input unit 112. The initial template amount value converting unit 102c may store the determined relational expression in the relational expression file 106c, and convert the amplification amount equivalent or the initial template amount equivalent N₀ to a value indicating the amplification amount or the initial template amount.

Preparative Method (I): a nucleic acid amplification reaction of a standard sample having the same target nucleic acid as the test sample is performed under the same condition as the test sample, an amplification amount (concentration or the like) of the amplification product of the target nucleic acid is measured, and a dilution series of the amplification product to which the intercalator is added is prepared under the same conditions as the test sample.

Preparative Method (II): a dilution series (a dilution series in which molar concentration or the like of the fluorescent molecule is specified) of a fluorescent molecule that emits the same signal as the reporter molecule that is added to the test sample is prepared.

Preparative Method (III): a nucleic acid amplification reaction of a standard sample having the same target nucleic acid as the test sample and having the added reporter molecule, is performed under the same condition as the test sample, an amplification amount (concentration or the like) of the amplification product of the target nucleic acid is measured, and a dilution series of the amplification product is prepared under the same conditions as the test sample.

An example of the processing of the target nucleic acid measuring apparatus 100 has been described hereinbefore. In the above explanation, the one example of preparing a dilution series in which the concentration is specified has been explained, but Embodiment 2 is not intended to be limited thereto. For example, a relationship expression may also be created by preparing a dilution series specifying on a unit such as copy number, mass, or amount of substance, according to the relationship between a label material such as an intercalator or a reporter molecule, and the template amount of nucleic acid (for example, one-on-one relationship as molar proportion).

According to Embodiment 2, the theoretical expression is fitted to the amplification amount equivalent measured at each thermal cycle number in the nucleic acid amplification reaction. Then, the initial template amount equivalent N₀ is calculated from the fitted theoretical expression, and the calculated initial template amount equivalent N₀ is converted to a value indicating the initial template amount. The theoretical expression includes an environmental coefficient K as an exponent parameter, and a parameter of an initial template amount equivalent N₀, and further includes at least one parameter among a saturation amount equivalent Nₘₐₓ, a reaction acceleration coefficient ρ, and a reaction inhibition coefficient μ. Therefore, according to Embodiment 2, a value indicating the initial template amount can be precisely determined using a theoretical expression that allows to be fitted directly to the measured amplification amount equivalent and is capable of precisely reflecting the actual amplification efficiency.

According to Embodiment 2, an amplification efficiency of the nucleic acid amplification reaction is defined in the theoretical expression by the environmental coefficient K and at least one parameter among the saturation amount equivalent Nₘₐₓ, the reaction acceleration coefficient ρ, and the reaction inhibition coefficient μ. Therefore, according to Embodiment 2, a value indicating the initial template amount can be more precisely determined using the theoretical expression that is capable of more precisely reflecting the actual amplification efficiency.

According to Embodiment 2, the theoretical expression expresses that the amplification amount equivalent is equal to the product of the initial template amount equivalent N₀ and a sequence of the amplification efficiencies to each of which 1 is added for each thermal cycle number. Therefore, according to Embodiment 2, a value indicating the initial template amount can be more precisely determined using the theoretical expression that allows to be fitted directly to the measured amplification amount equivalent and is capable of more precisely reflecting the actual amplification efficiency.

According to Embodiment 2, the theoretical expression is represented by the following expression, thereby enabling to determine a value indicating the initial template amount more precisely using the theoretical expression that is capable of more precisely reflecting the actual amplification efficiency.

$$Nn = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

According to Embodiment 2, the theoretical expression is fitted using the least square method, thereby enabling to perform the fitting and determine a value indicating the initial template amount, more precisely by fixing the obtained value of the parameter.

According to Embodiment 2, any one of the reaction acceleration coefficient ρ and the reaction inhibition coefficient μ or both are calculated to be fixed by fitting the theoretical expression to the amplification amount equivalent measured in a region where an increasing rate represented by the following expression is from 0 to 1. Therefore, according to Embodiment 2, the initial template amount equivalent can be more precisely determined only using an appropriate region of the measured amplification amount equivalents.

$${Δ}_{n} = \frac{{N}_{n}}{{N}_{n - 1}} - 1$$

(Here, Δₙ is the increasing rate; and Nₙ is the amplification amount equivalent at thermal cycle number n.)

According to Embodiment 2, a measurement value of a signal from an intercalator that is added to the test sample, and emits the signal only in the presence of a double-strand DNA but not in the absence of the double-strand DNA, is used as the amplification amount equivalent. In this case, a relational expression between the measurement value measured under the same condition as the test sample with respect to a dilution series prepared by the following preparative method (I) and the concentration of the dilution series is determined, and the amplification amount equivalent or the initial template amount equivalent N₀ is converted to a value indicating the amplification amount or the initial template amount based on the determined relational expression. Therefore, according to Embodiment 2, a value indicating the initial template amount can be more precisely determined using an accurate relational expression.

Preparative Method (I): a nucleic acid amplification reaction of a standard sample having the same target nucleic acid as the test sample is performed under the same condition as the test sample, an amplification amount (concentration or the like) of the amplification product of the target nucleic acid is measured, and a dilution series of the amplification product to which the intercalator is added is prepared under the same conditions as the test sample.

According to Embodiment 2, a measurement value of a signal from a reporter molecule that is added to the test sample, and emits the signal according to an extension reaction during the nucleic acid amplification reaction is used as the amplification amount equivalent. In this case, a relational expression between the measurement value measured under the same condition as the test sample with respect to a dilution series prepared by the following preparative method (II) and the concentration of the dilution series is determined, and the amplification amount equivalent or the initial template amount equivalent N₀ is converted to a value indicating the amplification amount or the initial template amount based on the determined relational expression. Therefore, according to Embodiment 2, a value indicating the initial template amount can be more precisely determined using an accurate relational expression.

Preparative Method (II): a dilution series of a fluorescent molecule that emits the same signal as the reporter molecule that is added to the test sample is prepared.

According to Embodiment 2, a measurement value of a signal from a reporter molecule that is added to the test sample, and emits the signal according to an extension reaction during the nucleic acid amplification reaction is used as the amplification amount equivalent. In this case, a relational expression between the measurement value measured under the same condition as the test sample with respect to a dilution series prepared by the following preparative method (III) and the concentration of the dilution series is determined, and the amplification amount equivalent or the initial template amount equivalent N₀ is converted to a value indicating the amplification amount or the initial template amount based on the determined relational expression. Therefore, according to Embodiment 2, a value indicating the initial template amount can be more precisely determined using an accurate relational expression.

Preparative Method (III): a nucleic acid amplification reaction of a standard sample having the same target nucleic acid as the test sample and having the added reporter molecule, is performed under the same condition as the test sample, an amplification amount (concentration or the like) of the amplification product of the target nucleic acid is measured, and a dilution series of the amplification product is prepared under the same conditions as the test sample. Here, the explanation of an example of Embodiment 2 will be ended.

### Example 4

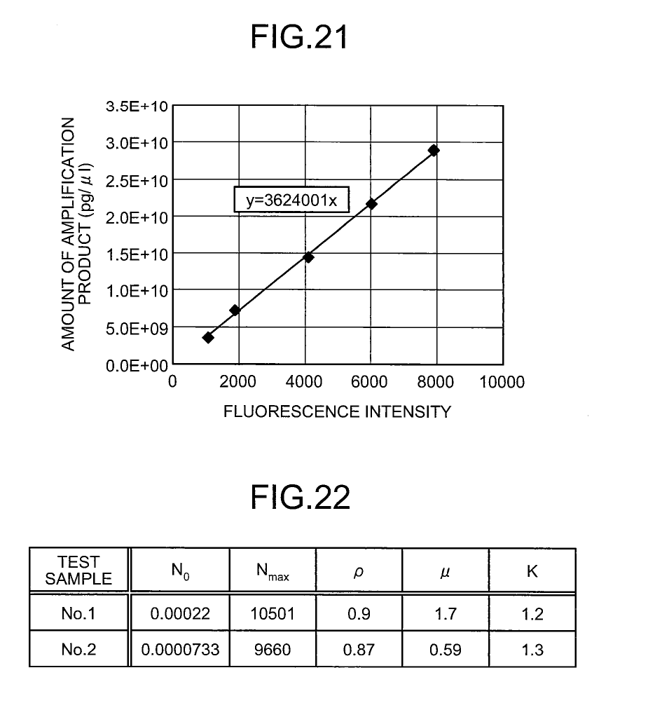

Example 4 according to Embodiment 2 will be described hereinafter with reference with FIGS. 21 to 35.

### Example 4-1

First, a measuring method of target nucleic acid according to real-time PCR using an intercalator will be explained below as Example 4-1.

(1) Relationship between Amplification Product Concentration and Fluorescence Intensity

Measurement conditions in Example 4-1 will be presented below. In this Example 4-1, a standard sample was prepared as follows by using primers, template DNA and reagents for human β-globin gene, which is the same target nucleic acid as a test sample.

### <Composition of Reaction Liquid>

primers for human β-globin: final concentration 0.3 μM each

2× Brilliant II SYBR Green qPCR master mix (trade name) (manufactured by Strategene, Inc.) as the reagents: 1× dilution concentration (final concentration)

Human Genomic DNA, Male (trade name) (manufactured by Promega Corporation, Cat# G1471) as the template DNA: final concentration 2000 pg/μl

The prepared standard sample was subjected to PCR under the following PCR conditions.

### <PCR Conditions>

qPCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

After purifying PCR product and removing the primers, absorbance of the amplification product was measured, and amplification product concentration was calculated based on the absorbance. Then, a dilution series was prepared so as to be similar composition with the above PCR reaction liquid, and fluorescence intensity was measured under the same condition as PCR of test sample. FIG. 21 is a diagram showing a relationship between amplification product concentration and fluorescence intensity. As a result, “y=3624001x” as shown in FIG. 21 was obtained as the relational expression between amplification product amount (x) and fluorescence intensity (y).

(2) Test Sample PCR

Test samples No.1 (prepared template amount: 800 pg/μl) and No.2 (prepared template amount: 250 pg/μl) were prepared under the following conditions.

### <Composition of Reaction Liquid>

primers for human β-globin: final concentration 0.3 μM each

2× Brilliant II SYBR Green qPCR master mix (trade name) (manufactured by Strategene, Inc.) as the reagents: 1× dilution concentration (final concentration)

Human Genomic DNA, Male (trade name) (manufactured by Promega Corporation, Cat# G1471) as the template DNA: final concentration 800 pg/μl in the test sample No.1, and 250 pg/μl in the test sample No.2

The test samples No.1 and No.2 were subjected to PCR under the following PCR conditions, and fluorescence intensity corresponding to thermal cycle number was measured to obtain amplification curve.

### <PCR Conditions>

qPCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

(3) Measurement of Initial Template Amount

The following theoretical expression was fitted to the obtained amplification curve to calculate values of the parameters for the test samples No.1 and No.2. FIG. 22 is a diagram showing calculation results of the parameters for the test samples No.1 and No.2.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent (the fluorescence intensity) at thermal cycle number n; N₀ is the initial template amount equivalent; j is thermal cycle number; Nⱼ is the amplification amount equivalent (the fluorescence intensity) at the thermal cycle number j; Nₘₐₓ is the saturation amount equivalent; ρ is the reaction acceleration coefficient; μ is the reaction inhibition coefficient; and K is the environmental coefficient.)

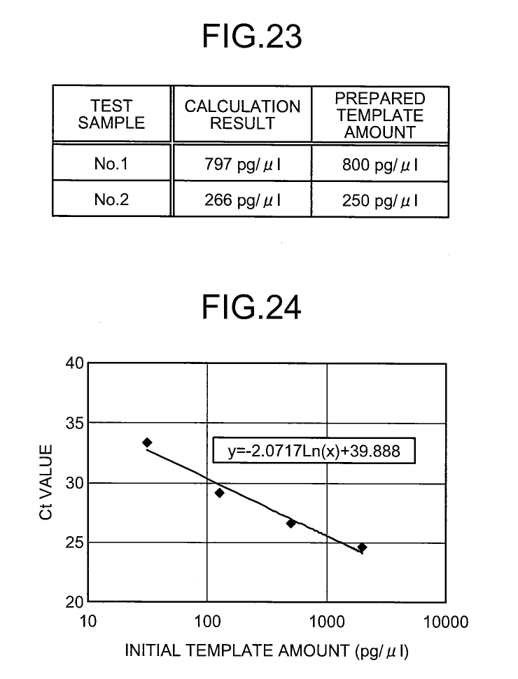

A value of the initial template amount equivalent N₀ shown in FIG. 22 is a value equivalent to the fluorescence intensity shown in FIG. 21. Consequently, the value of N₀ calculated using the theoretical expression from the amplification curve was assigned to x in “y=3624001x”, which is an approximate expression (relational expression) shown in FIG. 21 to calculate a value of the amplification product amount y, that is, a value indicating the initial template amount in the test sample. FIG. 23 is a diagram showing the calculation result of the initial template amount.

(4) Conventional Method

For comparison with the calculation result according to Example 4-1, the initial template amount was calculated for the test samples No.1 and No.2 using standard curve of Ct value according to the conventional method.

In order to create the standard curve of Ct value, standard samples were prepared by the above preparative method to be 2000 pg/μl, 500 pg/μl, 125 pg/μl, and 31.25 pg/μl as final concentrations of the initial template amounts, and the standard samples were subjected to PCR. Ct values were calculated using the software loaded on the qPCR apparatus, and standard curve of the Ct value versus the initial template amount was created. FIG. 24 is a diagram showing the standard curve of the Ct value versus the initial template amount.

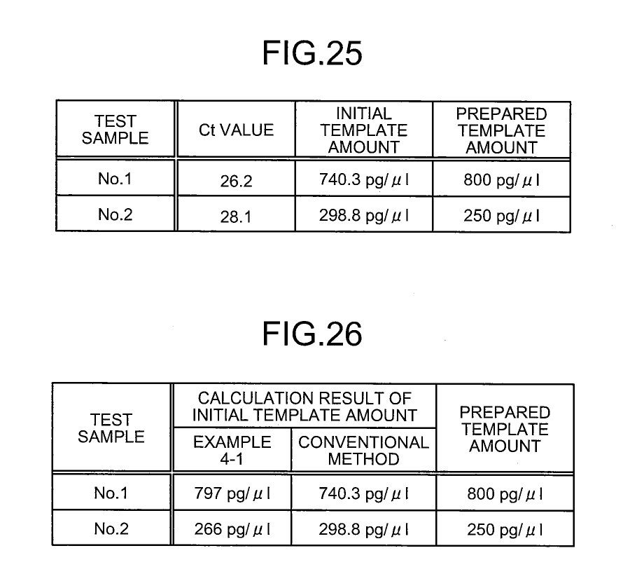

Subsequently, in order to quantify initial template amount of the test samples using the standard curve shown in FIG. 24, the above test samples No.1 (prepared template amount: 800 pg/μl) and No.2 (prepared template amount: 250 pg/μl) were subjected to PCR to calculate Ct value. Each initial template amount of the test samples was calculated from the calculated Ct value using the standard curve shown in FIG. 24. FIG. 25 is a diagram showing a calculation result of the initial template amount using the standard curve of Ct value.

(5) Comparison of the Results

As described above, the initial template amounts (pg/μl) were calculated according to Example 4-1 and the conventional example. FIG. 26 is a diagram showing comparison between the calculation results of the initial template amounts according to Example 4-1 and the conventional example.

As shown in FIG. 26, it was confirmed that the initial template amount calculated according to Example 4-1 represents the value when prepared (the prepared template amount) with similar or higher accuracy compared to the conventional method. According to Example 4-1, it was confirmed that an initial template amount of the test sample can be precisely calculated only using one standard sample for PCR without standard curve requiring that a large number of standard samples be subjected to PCR like conventional methods. In Example 4-1, the standard sample of which initial template amount had been known was used, but that of which initial template amount had not been known may also be used. In addition, in Example 4-1, the calculated value of the initial template amount equivalent N₀ was converted to the value indicating the initial template amount of the target nucleic acid, but the embodiment is not intended to be limited thereto. For example, after converting the amplification amount equivalent Nₙ at thermal cycle number n to a value (unit) indicating an initial template amount, the value is calculated to be N₀, that is, a value indicating the initial template amount, thereby enabling to obtain the same result.

### Example 4-2

Next, a measuring method of target nucleic acid according to real-time PCR using TaqMan (registered trademark) probe will be explained below as Example 4-2. (1) Relationship between Concentration of Fluorescent Substance and Fluorescence Intensity

A reporter molecule in TaqMan (registered trademark) probe, which is a PCR reagent of a test sample used in Example 4-2 is a fluorescent dye FAM (hereinafter, referred to as “FAM”). Since the number of molecules of target nucleic acid amplified by PCR in the test sample is equal to the number of molecules of the excited FAM, fluorescence intensity of FAM is equivalent to an amplification amount at thermal cycle number n.

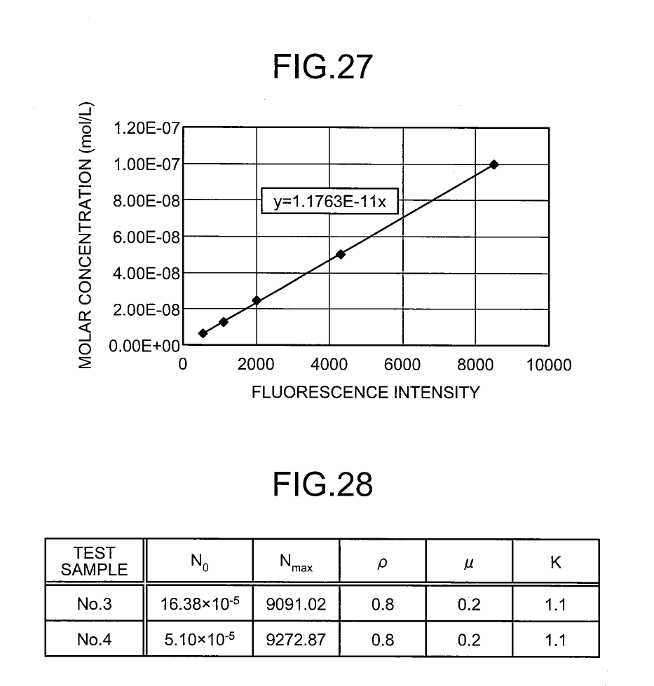

A dilution series of FAM was created to be similar composition with PCR reaction liquid, and fluorescence intensity was measured under the same conditions as PCR of the test sample. FIG. 27 is a diagram showing a relationship between molar concentration of FAM molecule and fluorescence intensity. As a result, “y=1.1763×10⁻¹¹×x” as shown in FIG. 27 was obtained as the relational expression between molar concentration of FAM molecule (y) and fluorescence intensity (x).

(2) Test Sample PCR

Test samples No.3 (prepared template amount: 800 pg/μl) and No.4 (250 pg/μl) were prepared under the following conditions.

### <Composition of Reaction Liquid>

primers for human β-globin: final concentration 0.3 μM each

TaqMan (registered trademark) probe (FAM-TAMRA (trade name)) as the probe: final concentration 0.2 μM

reagents: 2× Universal PCR Master Mix (trade name) (manufactured by Applied Biosystems, Inc.)

Human Genomic DNA, Male (trade name) (manufactured by Promega Corporation, Cat# G1471) as the template DNA: final concentration 800 pg/μl in the test sample No.3, and 250 pg/μl in the test sample No.4

The test samples No.3 and No.4 were subjected to PCR under the following PCR conditions.

### <PCR Conditions>

qPCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

(3) Measurement of Initial Template Amount

The following expression was fitted to the obtained amplification curve to calculate a value of each of the parameters for the test samples No.3 and No.4. FIG. 28 is a diagram showing the calculation result of the parameters for the test samples No.3 and No.4.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent (fluorescence intensity) at thermal cycle number n; N₀ is the initial template amount equivalent; j is thermal cycle number; Nⱼ is the amplification amount equivalent (fluorescence intensity) at the thermal cycle number j; Nₘₐₓ is the saturation amount equivalent; ρ is the reaction acceleration coefficient; μ is the reaction inhibition coefficient; and K is the environmental coefficient.)

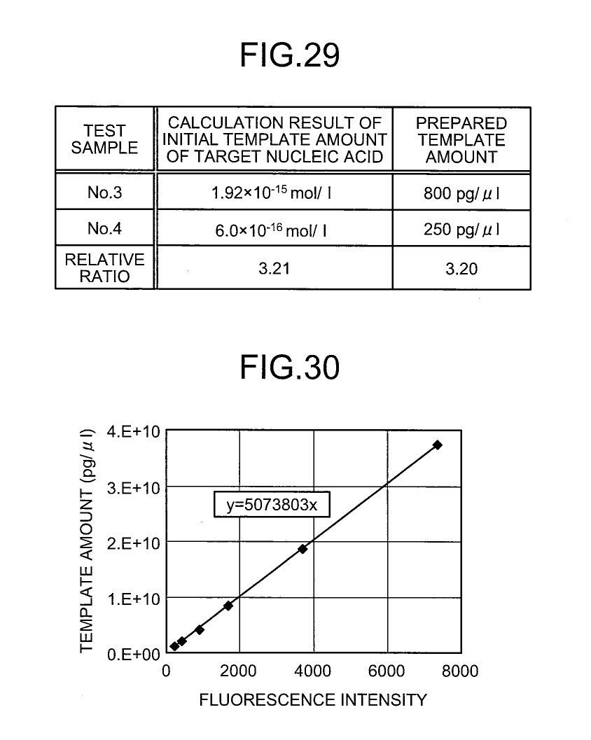

A value of the initial template amount equivalent N₀ shown in FIG. 28 is a value equivalent to the fluorescence intensity shown in FIG. 27. Consequently, the value of N₀ calculated using the theoretical expression from the amplification curve was assigned to x in “y=1.1763×10⁻¹¹×x”, which is an approximate expression (relational expression) as shown in FIG. 27 to calculate a value of the molar concentration y, that is, a value indicating the initial template amount in the test sample (molar concentration). FIG. 29 is a diagram showing the calculation result of the initial template amount. The relative ratio shown in FIG. 29 represents a relative ratio of a value of No.3 to a value of No.4.

(4) Comparison of the Results

As shown in FIG. 29, it was confirmed that the calculation result of the initial template amount calculated in Example 4-2 shows good correlation with the prepared template amount of the test sample (the calculation result 3.21 compared to a relative ratio of the prepared template amount, 3.20). According to Example 4-2, it was confirmed that an initial template amount of the test sample can be precisely calculated only by obtaining relationship between concentration of the fluorescent substance, which is a reporter molecule, and fluorescence intensity without need of PCR to which a large number of standard samples are subjected and also without standard curve like the conventional method. According to the conventional method, total nucleic acid amount in the test sample could be measured, but measurement of the target nucleic acid amount amplified actually was impossible, while use of Example 4-2 makes possible the measurement of the target nucleic acid amount.

### Example 4-3

Subsequently, another example of the measuring method of target nucleic acid according to real-time PCR using TaqMan (registered trademark) probe will be explained below as Example 4-3.

(1) Relationship Between Amplification Product Concentration and Fluorescence Intensity

Measurement conditions in Example 4-3 will be presented below. In Example 4-3, a standard sample was prepared as follows by using primers, template DNA and reagents for human β-globin gene, which is the same target nucleic acid as a test sample.

### <Composition of Reaction Liquid>

primers for human β-globin: final concentration 0.3 μM each

TaqMan (registered trademark) probe (FAM-TAMRA (trade name)) as the probe: final concentration 0.2 μM reagents: 2× Universal PCR Master Mix (trade name) (manufactured by Applied Biosystems, Inc.)

Human Genomic DNA, Male (trade name) (manufactured by Promega Corporation, Cat# G1471) as the template DNA: final concentration 2000 pg/μl

The prepared standard sample was subjected to PCR under the following PCR conditions.

### <PCR Conditions>

qPCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

After purifying PCR product and removing the primers, absorbance of the amplification product was measured, and amplification product concentration was calculated based on the absorbance. Then, a dilution series was prepared so as to be similar composition with the above PCR reaction liquid, and fluorescence intensity was measured under the same condition as PCR of test sample. FIG. 30 is a diagram showing a relationship between amplification product amount (template amount) and fluorescence intensity. As a result, “y=5073803x” as shown in FIG. 30 was obtained as the relational expression between template amount (x) and fluorescence intensity (y).

(2) Test Sample PCR

Test samples No.5 (prepared template amount: 800 pg/μl) and No.6 (prepared template amount: 250 pg/μl) were prepared under the following conditions.

### <Composition of Reaction Liquid>

primers for human β-globin: final concentration 0.3 μM each

TaqMan (registered trademark) probe (FAM-TAMRA (trade name)) as the probe: final concentration 0.2 μM reagents: 2× Universal PCR Master Mix (trade name) (manufactured by Applied Biosystems, Inc.)

The template DNA: final concentration 800 pg/μl in the test sample No.5, and 250 pg/μl in the test sample No.6

The test samples No.5 and No.6 were subjected to PCR under the following PCR conditions, and fluorescence intensity corresponding to thermal cycle number was measured to obtain amplification curve.

### <PCR Conditions>

qPCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

(3) Measurement of Initial Template Amount

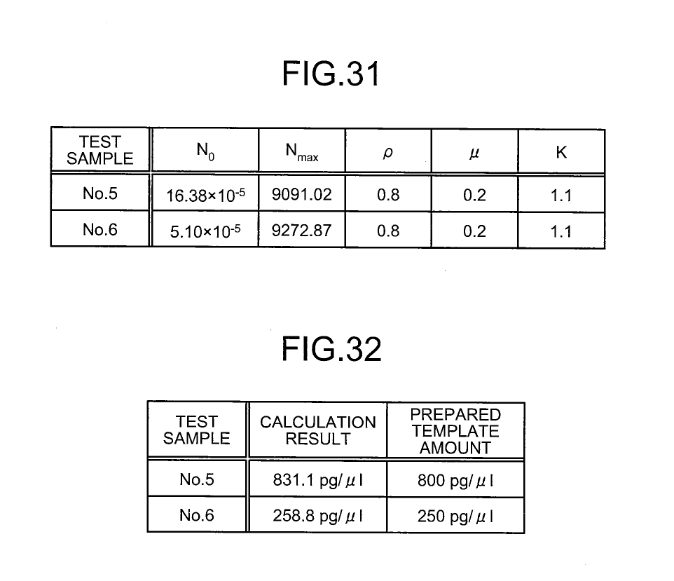

The following theoretical expression was fitted to the obtained amplification curve to calculate values of the parameters for the test samples No.5 and No.6. FIG. 31 is a diagram showing calculation results of the parameters for the test samples No.5 and No.6.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent (the fluorescence intensity) at thermal cycle number n; N₀ is the initial template amount equivalent; j is thermal cycle number; Nⱼ is the amplification amount equivalent (the fluorescence intensity) at the thermal cycle number j; Nₘₐₓ is the saturation amount equivalent; ρ is the reaction acceleration coefficient; μ is the reaction inhibition coefficient; and K is the environmental coefficient.)

A value of the initial template amount equivalent N₀ shown in FIG. 31 is a value equivalent to the fluorescence intensity shown in FIG. 30. Consequently, the value of N₀ calculated using the theoretical expression from the amplification curve was assigned to x in “y=5073803x”, which is an approximate expression (relational expression) as shown in FIG. 30 to calculate a value of the template amount y, that is, a value indicating the initial template amount in the test sample. FIG. 32 is a diagram showing the calculation result of the initial template amount.

(4) Conventional Method

For comparison with the calculation result according to Example 4-3, the initial template amount was calculated for the test samples No.5 and No.6 using standard curve of Ct value according to the conventional method.

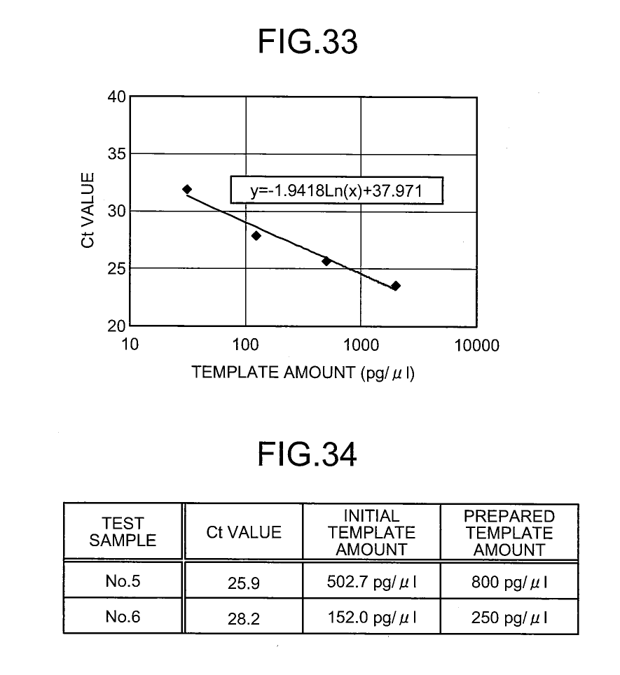

In order to create the standard curve of Ct value, standard samples were prepared by the above preparative method to be 2000 pg/μl, 500 pg/μl, 125 pg/μl, and 31.25 pg/μl as final concentrations of the initial template amounts, and the standard samples were subjected to PCR. Ct values were calculated using the software loaded on the qPCR apparatus, and standard curve of the Ct value versus the initial template amount was created. FIG. 33 is a diagram showing the standard curve of the Ct value versus the template amount.

Subsequently, in order to quantify initial template amount of the test samples using the standard curve shown in FIG. 33, the above test samples No.5 (prepared template amount: 800 pg/μl) and No.6 (prepared template amount: 250 pg/μl) were subjected to PCR to calculate Ct value. Each initial template amount of the test samples was calculated from the calculated Ct value using the standard curve shown in FIG. 33. FIG. 34 is a diagram showing a calculation result of the initial template amount using the standard curve of Ct value as the conventional example.

(5) Comparison of the Results

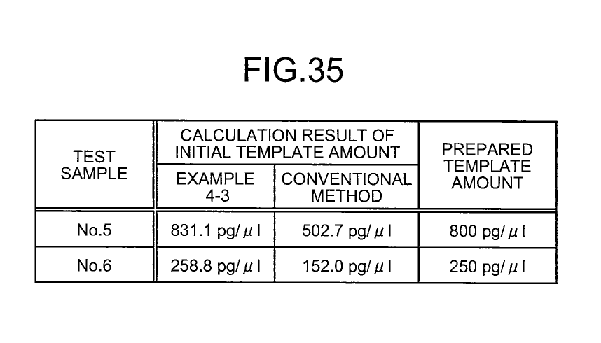

As described above, the initial template amounts (pg/μl) were calculated according to Example 4-3 and the conventional example. FIG. 35 is a diagram showing comparison between the calculation results of the initial template amounts according to Example 4-3 and the conventional example.

As shown in FIG. 35, it was confirmed that the initial template amount calculated according to Example 4-3 represents the value when prepared (the prepared template amount) with similar or higher accuracy compared to the conventional method. According to Example 4-3, it was confirmed that an initial template amount of the test sample can be precisely calculated only using one standard sample for PCR without standard curve requiring that a large number of standard samples be subjected to PCR like conventional methods. In Example 4-3, the standard sample of which initial template amount had been known was used, but that of which initial template amount had not been known may be also used. In addition, in Example 4-3, the calculated value of the initial template amount equivalent N₀ was converted to the value indicating the initial template amount of the target nucleic acid, but the embodiment is not intended to be limited thereto. For example, after converting the amplification amount equivalent at thermal cycle number n to a value indicating the amplification amount in the unit of the initial template amount, the value is calculated to be N₀, that is, a value indicating the initial template amount, thereby enabling to obtain the same result.

Thus, the explanation of Example 4 will be ended. In the Example 4, the relationship expression was derived under the premise that the relationship between the amplification amount equivalent (fluorescence intensity) and the template amount is linearly expressed. However, the relationship is not intended to be limited thereto, but a non-linear relationship expression might be derived as far as it gives better approximation.

### Embodiment 3

### Overview of Embodiment 3

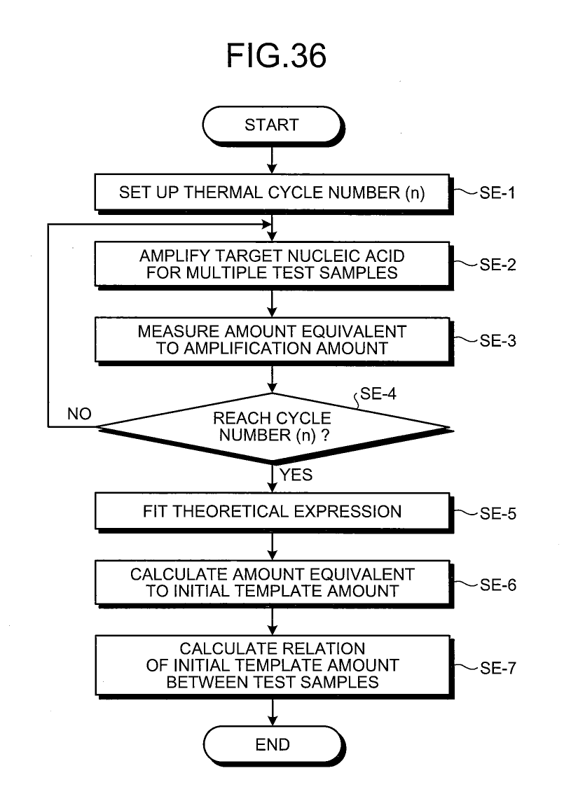

Hereinafter, an overview of Embodiment 3 will be explained with reference to FIG. 36, and then a configuration and processing of Embodiment 3 will be explained in detail. FIG. 36 is a flowchart for explaining an overview of Embodiment 3.

As shown in FIG. 36, according to Embodiment 3, first, the thermal cycle number (n) of a nucleic acid amplification reaction is set up (step SE-1). That is, the thermal cycle number (n) is set up for repeatedly performing the nucleic acid amplification reaction.

According to Embodiment 3, the nucleic acid amplification reaction of multiple test samples having the same target nucleic acid is performed to amplify the target nucleic acid (step SE-2). Here, at least one of the test samples may be a standard sample of which an initial template amount is known.

According to Embodiment 3, an amount equivalent to an amplification amount of the target nucleic acid (hereinafter, referred to as “amplification amount equivalent”) is measured corresponding to the thermal cycle number (step SE-3). Here, the “amplification amount” means the total amount of the target nucleic acid amplified from thermal cycle number 1 to the thermal cycle number when measuring. Also, the “amplification amount equivalent” is an amount equivalent to the amplification amount of the target nucleic acid, obtained by an arbitrary label. For example, the “amplification amount equivalent” is a signal intensity detected from an intercalator that emits the signal in the presence of a double-strand DNA, or a signal intensity detected from a reporter molecule that emits the signal according to an extension reaction during the amplification reaction.

According to Embodiment 3, it is determined whether current thermal cycle number has reached the set thermal cycle number (n) (step SE-4), and when the current thermal cycle number has not reached the set thermal cycle number (n) (step SE-4, No), the process is returned to the step SE-2. On the other hand, when the current thermal cycle number has reached the set thermal cycle number (n) (step SE-4, Yes), the nucleic acid amplification reaction and the measurement of the amplification amount equivalent according the reaction are finished.

According to Embodiment 3, a theoretical expression is fitted to the amplification amount equivalent measured in each thermal cycle (step SE-5). The theoretical expression according to Embodiment 3 includes an environmental coefficient as an exponent parameter, and a parameter of an amount equivalent to an initial template amount in the test sample (hereinafter, referred to as “initial template amount equivalent”). Further, this theoretical expression includes at least one parameter among an amount equivalent to a saturation amount upon the target nucleic acid amplification, a reaction acceleration coefficient, and a reaction inhibition coefficient. That is, according to Embodiment 3, the parameters of the theoretical expression are determined in this step SE-5 so as to fit the theoretical expression to the amplification amount equivalent measured in each thermal cycle.

In the theoretical expression, an amplification efficiency of the nucleic acid amplification reaction may be defined by the environmental coefficient and at least one parameter among the amount equivalent to the saturation amount upon the target nucleic acid amplification, the reaction acceleration coefficient, and the reaction inhibition coefficient. The theoretical expression may express that the amplification amount equivalent is equal to the product of the initial template amount equivalent and a sequence of the amplification efficiencies to each of which 1 is added for each thermal cycle number. According to Embodiment 3, at least one of the parameters may be a fixed value. According to Embodiment 3, when using the standard sample of which the initial template amount is known for at least one of the test samples, the theoretical expression may be fitted to the amplification amount equivalent measured at each thermal cycle number in the nucleic acid amplification reaction to which the standard sample is subjected. Then, at least one of the parameters may be calculated by the fitting of the theoretical expression, and the calculated parameter may be fixed to calculate the initial template amount for each of the test samples.

According to Embodiment 3, the initial template amount equivalent (that is, an amount equivalent to the initial template amount in the amplification reaction mixture at thermal cycle number 0) is calculated from the theoretical expression fitted to the amplification amount equivalent (step SE-6). For example, according to Embodiment 3, the initial template amount equivalent is calculated based on the theoretical expression in which the parameters have been determined by fitting.

According to Embodiment 3, a relation (ratio, proportions, magnitude correlation, or the like) of the initial template amount between the test samples is calculated by comparing the initial template amount equivalents calculated for each of the test samples (step SE-7). According to Embodiment 3, when using the standard sample of which the initial template amount is known for at least one of the test samples, a ratio of the initial template amount equivalent of each of the test samples to that of the standard sample may be calculated as the relation of the initial template amount between the test samples. And then, the initial template amount may be further calculated for each of the test samples by multiplying the calculated ratio by the initial template amount of the standard sample.

An overview of Embodiment 3 has been explained hereinbefore. According to Embodiment 3, the relation of the initial template amount between the test samples can be precisely determined using a theoretical expression that allows to be fitted directly to the measured amplification amount equivalent and is capable of precisely reflecting the actual amplification efficiency.

Configuration of Target Nucleic Acid Measuring Apparatus

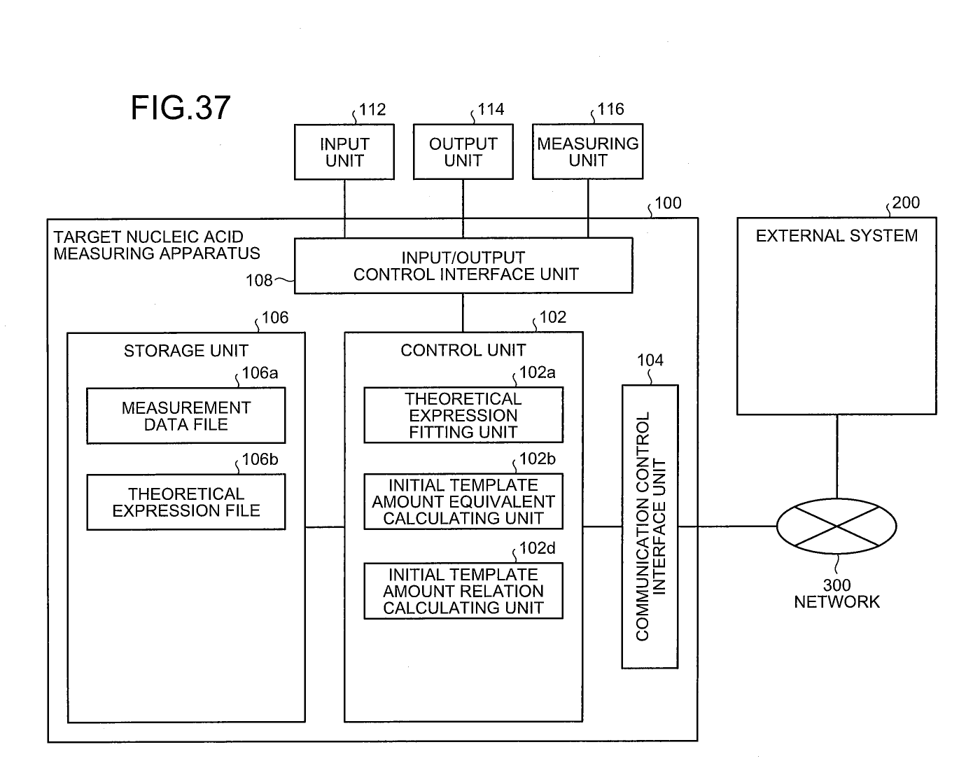

Next, a configuration of a target nucleic acid measuring apparatus according to Embodiment 3 will be explained below with reference to FIG. 37. FIG. 37 is a block diagram showing an example of a configuration of a target nucleic acid measuring apparatus 100 according to Embodiment 3 to which the present invention is applied, and schematically depicts only a part related to Embodiment 3 in the configuration.

In FIG. 37, the target nucleic acid measuring apparatus 100 schematically includes a control unit 102, a communication control interface unit 104, an input/output control interface unit 108, and a storage unit 106. The control unit 102 is a CPU and the like that integrally controls the entire operation of the target nucleic acid measuring apparatus 100. The input/output control interface unit 108 is an interface connected to an input unit 112, an output unit 114, and a measuring unit 116. The storage unit 106 is a device that stores various databases, tables or the like. These components of the target nucleic acid measuring apparatus 100 are communicably connected through an arbitrary communication path.

The various databases or files (a measurement data file 106a, and a theoretical expression file 106b) stored in the storage unit 106 are storage means such as a fixed disk device. For example, the storage unit 106 stores various programs, tables, files, databases and the like which are used in various processes.

Of these constituent elements of the storage unit 106, the measurement data file 106a stores an amplification amount equivalent of the target nucleic acid measured at each thermal cycle number, corresponding to the thermal cycle number in the nucleic acid amplification reaction (for example, PCR) for multiple test samples. That is the multiple test samples having the same target nucleic acid has been subjected to this nucleic acid amplification reaction. The measurement data file 106a may store measurement data on a standard sample of which the initial template amount is known as one of the test samples. That is, the measurement data file 106a may store the amplification amount measured at each thermal cycle number in the nucleic amplification reaction for the standard sample of which the initial template amount is known.

The theoretical expression file 106b stores a theoretical expression. The theoretical expression stored in the theoretical expression file 106b includes an environmental coefficient K as an exponent parameter, and a parameter of an initial template amount equivalent N₀. Further, the theoretical expression includes at least one parameter among an amount equivalent to a saturation amount upon the target nucleic acid amplification (hereinafter, referred to as “saturation amount equivalent”) Nₘₐₓ, a reaction acceleration coefficient ρ, and a reaction inhibition coefficient μ.

In the theoretical expression stored in the theoretical expression file 106b, an amplification efficiency of the nucleic acid amplification reaction may be defined by the environmental coefficient K and at least one parameter among the saturation amount equivalent Nₘₐₓ, the reaction acceleration coefficient ρ, and the reaction inhibition coefficient μ. Further, the theoretical expression may express that the amplification amount equivalent Nₙ is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. For example, the theoretical expression may be represented by the following expression.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

The theoretical expression file 106b may store a value of the parameter in the theoretical expression. For example, the theoretical expression file 106b may store, as a fixed value, at least one value of the reaction acceleration coefficient ρ, the reaction inhibition coefficient μ, the environmental coefficient K, and the saturation amount equivalent Nₘₐₓ.

In FIG. 37, the input/output control interface unit 108 controls the input unit 112, the output unit 114, and the measuring unit 116. As the output unit 114, not only a monitor (including a household-use television) but also a speaker may be used (hereinafter, the output unit 114 is sometimes described as a monitor). As the input unit 112, a keyboard, a mouse device, a microphone or the like may be used.

The measuring unit 116 measures an amplification amount equivalent at each thermal cycle number, corresponding to the thermal cycle number in the nucleic acid amplification reaction to which the test samples having the same target nucleic acid are subjected. As an example, the measuring unit 116 is constituted as a measuring means in a real-time PCR apparatus or the like. In addition, the amplification amount equivalent measured by the measuring unit 116 for each thermal cycle number is stored as measurement data in the measurement data file 106a under the control of the control unit 102.

In FIG. 37, the control unit 102 has an internal memory to store a control program such as an OS (Operating System), a program that defines various procedures, and required data. The control unit 102 performs information processing to execute various processes by these programs or the like. The control unit 102 functionally conceptually includes a theoretical expression fitting unit 102a, an initial template amount equivalent calculating unit 102b, and an initial template amount relation calculating unit 102d.

The theoretical expression fitting unit 102a fits the theoretical expression stored in the theoretical expression file 106b, to the amplification amount equivalent for each thermal cycle number stored in the measurement data file 106a. That is, the theoretical expression fitting unit 102a determines values of the parameters in the theoretical expression so that the theoretical expression has the best fit to the amplification amount equivalent measured at each thermal cycle number.

Here, the theoretical expression fitting unit 102a may fit the theoretical expression using the least square method. The theoretical expression fitting unit 102a may read out the value of the parameters stored in the theoretical expression file 106b, such as the reaction acceleration coefficient ρ, the reaction inhibition coefficient μ, the environmental coefficient K, or the saturation amount equivalent Nₘₐₓ. And then, the theoretical expression fitting unit 102a may use a predetermined value such as the read-out value of at least one of these parameters as a fixed value when fitting the theoretical expression. For example, the theoretical expression fitting unit 102a may determine values of the parameters in the theoretical expression stored in the theoretical expression file 106b by fitting the theoretical expression to the amplification amount equivalent for the standard sample for each thermal cycle number stored in the measurement data file 106a, and store at least one of the determined parameters as a fixed value in the theoretical expression file 106b. That is, this value of the parameter stored in the theoretical expression file 106b is read out to fix the parameter when fitting the theoretical expression for the measurement data of each of the test samples. The theoretical expression fitting unit 102a may perform the fitting of the theoretical expression by setting the maximum value of the amplification amount equivalents stored in the measurement data file 106a as the saturation amount equivalent Nₘₐₓ. The theoretical expression fitting unit 102a may fix the parameter such as the reaction acceleration coefficient ρ or the reaction inhibition coefficient μ by fitting the theoretical expression to the amplification amount equivalent measured only in a region where the increasing rate Δₙ represented by the following expression is from 0 to 1. That is, the theoretical expression fitting unit 102a may fit the theoretical expression only to the amplification amount equivalent measured at the thermal cycle number of which the increasing rate Δₙ is from 0 to 1. And then, the theoretical expression fitting unit 102a may store, as a fixed value, the value of the parameter such as the reaction acceleration coefficient ρ or the reaction inhibition coefficient μ determined from the fitted theoretical expression, in the theoretical expression file 106b.

$${Δ}_{n} = \frac{{N}_{n}}{{N}_{n - 1}} - 1$$

(Here, Δₙ is the increasing rate; and Nₙ is the amplification amount equivalent at thermal cycle number n.)

The initial template amount equivalent calculating unit 102b calculates the initial template amount equivalent from the theoretical expression fitted by the theoretical expression fitting unit 102a. For example, the initial template amount equivalent calculating unit 102b calculates the initial template amount equivalent N₀ for each of the test samples based on the values of the parameters in the theoretical expression, which are the results of the fitting performed by the theoretical expression fitting unit 102a.

The initial template amount relation calculating unit 102d calculates a relation of the initial template amount between the test samples by comparing the initial template amount equivalents calculated by the initial template amount equivalent calculating unit 102b for each of the test samples. For example, the initial template amount relation calculating unit 102d calculates a relation of the initial template amount between the test samples based on the initial template amount equivalents calculated by the initial template amount equivalent calculating unit 102b for each of the test samples. For example, the initial template amount relation calculating unit 102d calculates the relation of the initial template amount between the test samples by calculating a rate, a ratio, a proportion, a magnitude correlation, or the like of the initial template amount equivalent between the test samples to output the relation to the output unit 114. The initial template amount relation calculating unit 102d may calculate a ratio of the initial template amount equivalent of the test sample to that of the standard sample as the relation of the initial template amount between the test samples, and calculate the initial template amount for each of the test samples by multiplying the calculated ratio by the initial template amount (known amount) of the standard sample.

An example of the configuration of the target nucleic acid measuring apparatus 100 has been explained hereinbefore. The target nucleic acid measuring apparatus 100 may be communicably connected to a network 300 through a communication device such as a router and a wired or radio communication line such as a leased line. In this case, the communication control interface unit 104 is an interface connected to a communication device (not shown) such as a router connected to the communication line or the like, and performs communication control between the target nucleic acid measuring apparatus 100 and the network 300 (or a communication device such as a router). Namely, the communication control interface unit 104 has a function of performing data communication with another terminal through a communication line. In FIG. 37, the network 300 has a function of connecting the target nucleic acid measuring apparatus 100 and an external system 200 with each other. For example, the Internet is used as the network 300.

The target nucleic acid measuring apparatus 100 may be connected to the external system 200 which provides an external program making a computer serve as the target nucleic acid measuring apparatus, or an external database related to the measurement data or the parameters, through the network 300.

In FIG. 37, the external system 200 is mutually connected to the target nucleic acid measuring apparatus 100 through the network 300. And the external system 200 has a function of providing an external database related to the measurement data, the theoretical expressions, or values of the parameters, and an external program such as a target nucleic acid measuring program that makes an information processing device serve as the target nucleic acid measuring apparatus, to a user. The external system 200 may be designed to serve as a WEB server or an ASP server. The hardware configuration of the external system 200 may be constituted by an information processing device such as a commercially available workstation or personal computer and a peripheral device thereof. The functions of the external system 200 are realized by a CPU, a disk device, a memory device, an input unit, an output unit, a communication control device, and the like in the hardware configuration of the external system 200 and programs which control these devices.

Processing of Target Nucleic Acid Measuring Apparatus 100

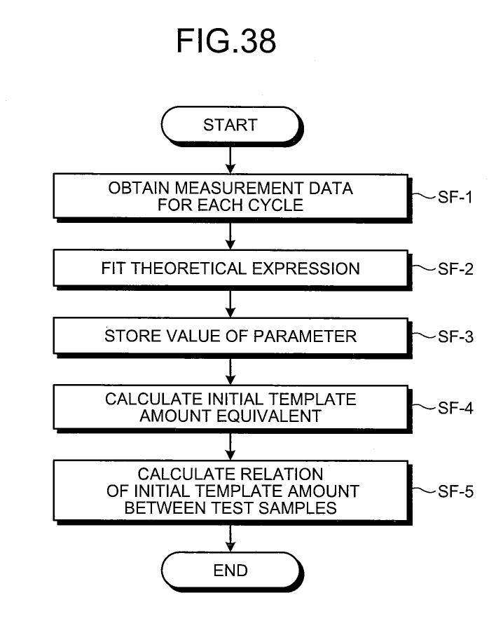

Next, an example of processing of the target nucleic acid measuring apparatus 100 according to Embodiment 3 constructed as described above will be explained below in detail with reference to FIG. 38. FIG. 38 is a flowchart showing an example of the processing of the target nucleic acid measuring apparatus 100 according to Embodiment 3.

The measuring unit 116 of a real-time PCR measuring apparatus or the like measures an amplification amount equivalent (for example, a measurement value of a signal from the intercalator or the reporter molecule) at each thermal cycle number, corresponding to the thermal cycle number in the nucleic acid amplification reaction to which multiple test samples having the same target nucleic acid are subjected. As shown in FIG. 38, the control unit 102 of the target nucleic acid measuring apparatus 100 obtains measurement data on the amplification amount equivalent measured at each thermal cycle number through the measuring unit 116, and stores the measurement data in the measurement data file 106a (step SF-1). Here, at least one of the test samples may be a standard sample of which an initial template amount is known.

The theoretical expression fitting unit 102a fits the theoretical expression stored in the theoretical expression file 106b, to the amplification amount equivalent for each thermal cycle number stored in the measurement data file 106a (step SF-2). That is, the theoretical expression fitting unit 102a adjusts and determines values of the parameters in the theoretical expression using the least square method so that the theoretical expression has the best fit to the amplification amount equivalent for each thermal cycle number. In this theoretical expression, an amplification efficiency of the nucleic acid amplification reaction may be defined by not only the environmental coefficient K, but also a parameter such as the saturation amount equivalent Nₘₐₓ, the reaction acceleration coefficient ρ, or the reaction inhibition coefficient μ. As an example, the theoretical expression expresses that the amplification amount equivalent is equal to the product of the initial template amount equivalent N₀ and a sequence of the amplification efficiencies to each of which 1 is added for each thermal cycle number. That is, the theoretical expression may express that the amplification amount equivalent Nₙ is equal to the product of the initial template amount equivalent N₀ and a sequence (Eₖ+1) (k=1, 2, . . . n), wherein Eₖ denotes the amplification efficiency at thermal cycle number k. Exemplarily, the theoretical expression is represented by the following expression.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

In the step SF-2, the theoretical expression fitting unit 102a may read out from the theoretical expression file 106b the value of the parameters such as the reaction acceleration coefficient ρ, the reaction inhibition coefficient μ, the environmental coefficient K, or the saturation amount equivalent Nₘₐₓ. And then, the theoretical expression fitting unit 102a may use a predetermined value such as the read-out value of any of these parameters as a fixed value common to the test samples. For example, the theoretical expression fitting unit 102a may determine values of the parameters in the theoretical expression by fitting the theoretical expression to the amplification amount equivalent of the standard sample for each thermal cycle number stored in the measurement data file 106a. Then, the theoretical expression fitting unit 102a may fit the theoretical expression in which at least one value of the determined parameters is fixed to the amplification amount equivalent of the test sample for each thermal number.

The theoretical expression fitting unit 102a stores each value of the parameters of the fitted theoretical expression, in the theoretical expression file 106b (step SF-3). For example, the theoretical expression fitting unit 102a stores, as a result of the fitting, those values of the parameters optimized so that the theoretical expression has the best fit to the amplification amount equivalent using the least square method or the like, in the theoretical expression file 106b.

The initial template amount equivalent calculating unit 102b calculates the initial template amount equivalent N₀ for each of the test samples based on the values of the parameters of the theoretical expression, stored in the theoretical expression file 106b (step SF-4).

The initial template amount relation calculating unit 102d calculates a relation (a relative quantitative value such as rate, ratio, proportion, magnitude correlation, or the like) of the initial template amount between the test samples by comparing the initial template amount equivalents calculated by the initial template amount equivalent calculating unit 102b for each of the test samples. And then, the initial template amount relation calculating unit 102d outputs the calculated relation of the initial template amount between the test samples to the output unit 114 through the input/output control interface unit 108 (step SF-5). The initial template amount relation calculating unit 102d may calculate a ratio of the initial template amount equivalent of the test sample to that of the standard sample as the relation of the initial template amount between the test samples, and calculate the initial template amount for each of the test samples by multiplying the calculated ratio by the initial template amount (known amount) of the standard sample. An example of the processing of the target nucleic acid measuring apparatus 100 has been described hereinbefore.

According to Embodiment 3, the theoretical expression is fitted to the amplification amount equivalent measured at each thermal cycle number in the nucleic acid amplification reaction to which multiple test samples having the same target nucleic acid are subjected. And the initial template amount equivalent N₀ is calculated from the fitted theoretical expression, and a relation of the initial template amount between the test samples is calculated by comparing the initial template amount equivalents N₀ calculated for each of the test samples. The theoretical expression includes an environmental coefficient K as an exponent parameter, and a parameter of an initial template amount equivalent N₀, and further includes at least one parameter among a saturation amount equivalent Nₘₐₓ a reaction acceleration coefficient ρ, and a reaction inhibition coefficient μ. Therefore, according to Embodiment 3, a relation of the initial template amount between the test samples can be precisely determined using a theoretical expression that allows to be fitted directly to the measured amplification amount equivalent and is capable of precisely reflecting the actual amplification efficiency.

According to Embodiment 3, an amplification efficiency of the nucleic acid amplification reaction is defined in the theoretical expression by the environmental coefficient K and at least one parameter among the saturation amount equivalent Nₘₐₓ, the reaction acceleration coefficient ρ, and the reaction inhibition coefficient μ. Therefore, according to Embodiment 3, a relation of the initial template amount between the test samples can be more precisely determined using the theoretical expression that is capable of more precisely reflecting the actual amplification efficiency.

According to Embodiment 3, the theoretical expression expresses that the amplification amount equivalent is equal to the product of the initial template amount equivalent N₀ and a sequence of the amplification efficiencies to each of which 1 is added for each thermal cycle number. Therefore, according to Embodiment 3, a relation of the initial template amount between the test samples can be more precisely determined using the theoretical expression that allows to be fitted directly to the measured amplification amount equivalent and is capable of more precisely reflecting the actual amplification efficiency.

According to Embodiment 3, the theoretical expression is represented by the following expression, thereby enabling to determine a relation of the initial template amount between the test samples more precisely using the theoretical expression that is capable of more precisely reflecting the actual amplification efficiency.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.) According to Embodiment 3, the theoretical expression is fitted using the least square method, thereby enabling to perform the fitting and determine a relation of the initial template amount between the test samples, more precisely by fixing the obtained value of the parameter.

According to Embodiment 3, at least one of the parameters in the theoretical expression is fixed as a fixed value, thereby enabling to precisely determine a relation of the initial template amount between the test samples in a short amount of time.

According to Embodiment 3, any one of the reaction acceleration coefficient ρ and the reaction inhibition coefficient μ or both are calculated to be fixed by fitting the theoretical expression to the amplification amount equivalent measured in a region where an increasing rate represented by the following expression is from 0 to 1. Therefore, according to Embodiment 3, the initial template amount equivalent can be more precisely determined only using an appropriate region of the measured amplification amount equivalents.

$${Δ}_{n} = \frac{{N}_{n}}{{N}_{n - 1}} - 1$$

(Here, Δₙ is the increasing rate; and Nₙ is the amplification amount equivalent at thermal cycle number n.)

According to Embodiment 3, at least one of the test samples is a standard sample of which the initial template amount is known, and a ratio of the amount equivalent of each of the test samples to that of the standard sample is calculated. Then, the initial template amount is calculated for each of the test samples by multiplying the calculated ratio by the initial template amount of the standard sample. According to Embodiment 3, an initial template amount can be precisely determined for each of the test samples using the precisely-calculated relation of the initial template amount between the test samples.

According to Embodiment 3, the theoretical expression is fitted to the amplification amount equivalent measured for each thermal cycle number, corresponding to the thermal cycle number in the nucleic acid amplification reaction to which the standard sample is subjected. Then, at least one of the parameters in the theoretical expression is calculated by the fitting, and the calculated value is fixed as a fixed value to calculate an initial template amount for each of the test samples. Therefore, according to Embodiment 3, an initial template amount can be more precisely determined for each of the test samples by precisely calculating the relation of the initial template amount between the test samples using the fixed value common with the standard sample. Here, the explanation of an example of Embodiment 3 will be ended.

### Example 5

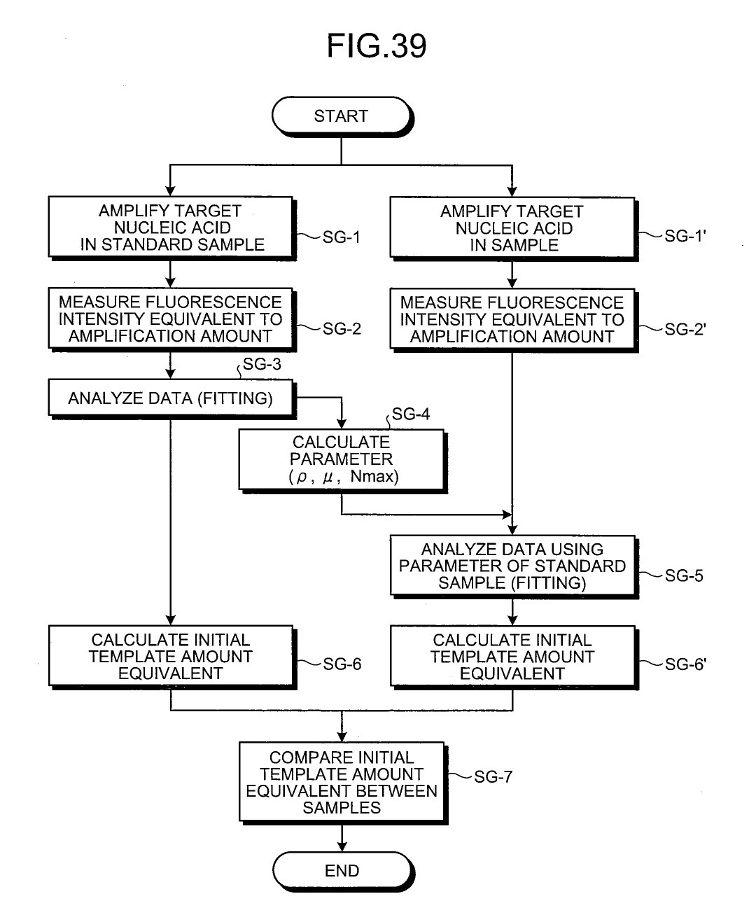

Example 5 according to Embodiment 3 will be explained hereinafter with reference with FIGS. 39 to 46.

First, an overview of Example 5 will be presented below. In Example 5, a ratio of the initial template amount equivalent was calculated to calculate the initial template amount as follows. FIG. 39 is a diagram showing a measuring method of the target nucleic acid according to Example 5.

As shown in FIG. 39, first, multiple test samples having the same target nucleic acid are subjected to nucleic acid amplification reaction to amplify the target nucleic acid in Example 5 (step SG-1′). Here, in Example 5, in order to calculate the initial template amount from a ratio of the initial template amount equivalent for the last time, a standard sample of which the initial template amount is known is subjected to the nucleic acid amplification reaction concurrently to amplify the target nucleic acid (step SG-1).

In Example 5, amplification amount equivalents (fluorescence intensity, in this Example 5) of the test sample and the standard sample is measured at each thermal cycle number, corresponding to the thermal cycle in the nucleic acid amplification reaction (step SG-2, SG-2′)

In Example 5, a theoretical expression is fitted to the measurement data of the standard sample (the amplification amount equivalents measured at each thermal cycle number) to conduct data analysis (that is, the fitting of the theoretical expression) (step SG-3). In this Example 5, the following expression developed by the inventors of the present application is used as a theoretical expression.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, Nₙ is the amplification amount equivalent at thermal cycle number n; N₀ is the initial template amount equivalent; j is thermal cycle number; Nⱼ is the amplification amount equivalent at the thermal cycle number j; Nₘₐₓ is the saturation amount equivalent; ρ is the reaction acceleration coefficient; μ is the reaction inhibition coefficient; and K is the environmental coefficient.)

In Example 5, the parameters of the reaction acceleration coefficient ρ, the reaction inhibition coefficient μ, and the saturation amount equivalent Nₘₐₓ are calculated (evaluated) as a result of the data analysis (the fitting result) for the measurement data of the standard sample (step SG-4).

In Example 5, the theoretical expression is fitted to the measurement data of each of the test samples using as fixed values the parameters calculated as the result of the data analysis of the standard sample to conduct data analysis (the fitting) (step SG-5). As such, in Example 5, in order to minimize errors in a result of comparing the initial template amount equivalents, the parameters in the theoretical expression are set as fixed values common to the standard sample and the test sample.

In Example 5, an initial template amount equivalent is calculated for the standard sample and the test sample based on the result of the data analysis (the fitting result) (step SG-6, SG-6′)

In Example 5, a ratio of the initial template amount is calculated by comparing the initial template amount equivalent between the standard sample and the test sample, and the initial template amount is calculated for each of the test samples by multiplying the calculated ratio of the initial template amount by the initial template amount of the standard sample (step SG-7). An overview of Example 5 has been explained hereinbefore.

Measurement conditions in Example 5 will be presented below. In Example 5, the test sample to be subjected to PCR as the nucleic acid amplification was prepared as follows by using primers, template DNA and reagents.

primers for human β-globin: final concentration 0.3 μM each

2× Brilliant II SYBR Green qPCR master mix (trade name) (manufactured by Strategene, Inc.) as the reagents: 1× dilution concentration (final concentration)

template DNA: Human Genomic DNA, Male (trade name) (manufactured by Promega Corporation, Cat# G1471)

The prepared test sample was subjected to PCR under the following conditions to obtain measurement data of cycle number versus fluorescence intensity.

real-time PCR apparatus: ABI Prism 7900HT (trade name) (manufactured by Applied Biosystems, Inc.)

PCR temperature conditions: 95° C./10 minutes→(95° C./30 seconds→60° C./1 minute)×40 cycles

For comparison with the calculation result according to Example 5, a ratio of the initial template amount between the test samples, which has different concentrations for each another, was calculated using Ct value according to the standard curve method and the comparative Ct method.

Standard Curve Method

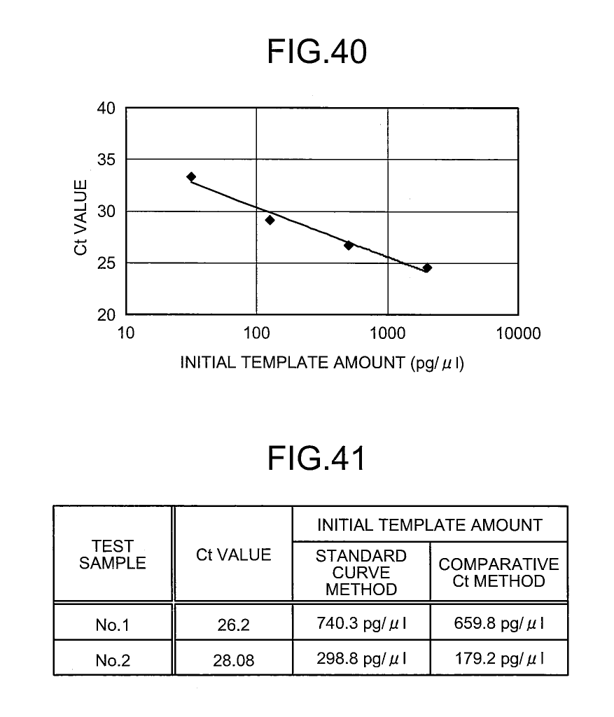

Standard samples were prepared to be 2000 pg/μl, 500 pg/μl, 125 pg/μl, and 31.25 pg/μl as final concentrations of the initial template amounts using the same reagents as the test sample of Example 5, and the standard samples were subjected to PCR. Ct values were calculated using the software loaded on the real-time PCR apparatus, and standard curve of the Ct value versus the initial template amount was created. FIG. 40 is a diagram showing the standard curve of the Ct value versus the initial template amount.

The test samples No.1 and No.2 having 800 pg/μl and 250 pg/μl of prepared initial template amounts were subjected to PCR to calculate Ct value. Each initial template amount of the test samples was calculated from the calculated Ct value using the standard curve shown in FIG. 40.

Comparative Ct Method

The initial template amounts of the test samples No.1 and No.2 were calculated using the following expression consisting of Ct value “24.6” at 2000 pg/μl of the initial template amount.

Ns=2000×2^{(24.6-Cts)}

(Ns is the initial template amount (pg/μl) of the test sample, and Cts is the Ct value of the test sample.)

As described above, the initial template amounts of the test samples No.1 and No.2 were calculated using Ct value according to the conventional methods, which are the standard curve method and the comparative Ct method. FIG. 41 is a diagram showing the calculation result according to the conventional examples, which are the standard curve method and the comparative Ct method.

### Example 5-1

As Example 5-1, the following expression was fitted to the amplification curve of the test samples No.1 and No.2, which is the same measurement data as that used by the conventional example.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, N₀ is the initial template amount equivalent; Nₙ is the fluorescence intensity as the amplification amount equivalent at thermal cycle number n; Nₘₐₓ is the saturation amount equivalent; ρ is the reaction acceleration coefficient; μ is the reaction inhibition coefficient; and K is the environmental coefficient.)

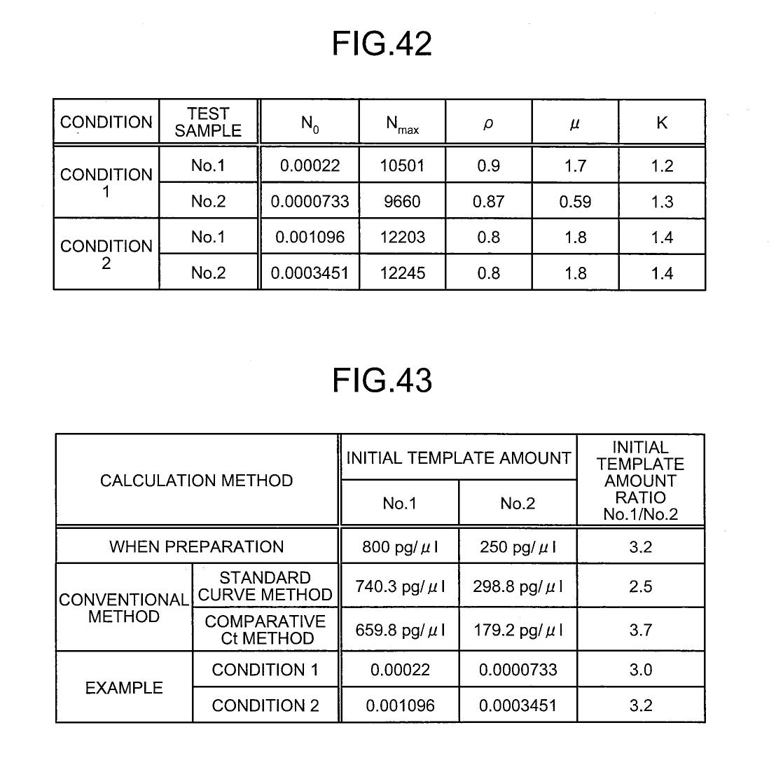

In Example 5-1, the fittings were performed in two cases where all of the parameters are not fixed as Condition 1, and where the environmental coefficient K is fixed as Condition 2. FIG. 42 is a diagram showing the calculation result of each of the parameters by fitting in the 2 cases of Condition 1 and Condition 2 for the test samples No.1 and No.2. From the fitting result shown in FIG. 42, a ratio of the initial template amount equivalent N₀ (initial template amount ratio) was calculated between the test samples No.1 and No.2.

As mentioned above, the initial template amount (equivalent) and the initial template amount ratio were calculated according to Example 5-1 and the conventional example. FIG. 43 is a diagram showing the initial template amount (equivalent) and the initial template amount ratio calculated according to Example 5-1 and the conventional example.

As shown in FIG. 43, according to Example 5-1, it was confirmed that a ratio of the initial template amount can be calculated without need of PCR to which a large number of standard samples are subjected and also without standard curve like the standard curve method. In addition, since the actual amplification ratio can be reflected more precisely compared with the comparative Ct method in which the fixed value, 2, is set as the amplification rate, a value extremely close to the ratio of the prepared initial template amount (=3.2) can be calculated, thereby enabling to calculate a ratio of the initial template amount with similar or higher accuracy compared to the conventional method. Also, it was confirmed that the theoretical expression can be fitted directly to the measurement result (the measurement data) without conversion to the initial template amount using standard curve.

### Example 5-2

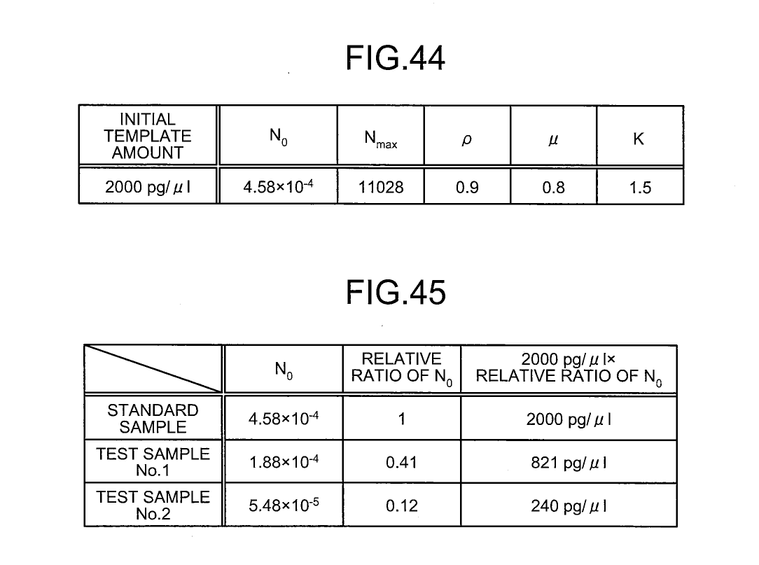

Subsequently, as Example 5-2, the following theoretical expression was fitted for the standard sample of which the initial template amount is 2000 pg/μl, measured in Example 5-1 to calculate each of the parameters. FIG. 44 is a diagram showing a value of each of the calculated parameters.

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(Here, N₀ is the initial template amount equivalent; Nₙ is the fluorescence intensity as the amplification amount equivalent at thermal cycle number n; Nₘₐₓ is the saturation amount equivalent; ρ is the reaction acceleration coefficient; μ is the reaction inhibition coefficient; and K is the environmental coefficient.)

Values of ρ, μ, and K is fixed to be 0.9, 0.8, and 1.8, respectively based the calculation result shown in FIG. 44, and each value of the test samples No.1 and No.2 was calculated using the theoretical expression. Then, a ratio (relative ratio) of the calculated initial template amount equivalent N₀ of the test sample to that of the standard sample was calculated, and the initial template amount was calculated by multiplying the ratio by 2000 pg/μl, which is the initial template amount of the standard sample, for each of the test samples No.1 and No.2. FIG. 45 is a diagram showing the initial template amount calculated for the test samples No.1 and No.2.

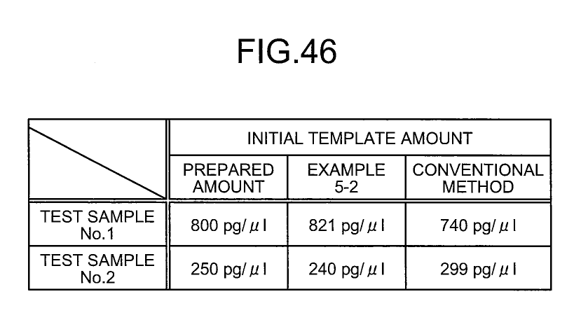

Comparison with the conventional method shown in Example 5-1 was made. FIG. 46 is a diagram showing a result of the comparison of the calculated initial template amount between Example 5-2 and the conventional method (standard curve method) for the test samples No.1 and No. 2.

As shown in FIG. 46, it was confirmed that the initial template amount calculated by Example 5-2 almost exactly reflects a value of when prepared with similar or higher accuracy compared to the conventional method. According to Example 5, it was confirmed that an initial template amount of the test sample can be precisely calculated only using one standard sample for PCR without need of PCR to which a large number of standard samples are subjected and also without standard curve. Also, it was confirmed that the theoretical expression can be fitted directly to the measurement result (the measurement data) without conversion to the initial template amount using standard curve.

As such, according to Example 5, a relation of the initial template amount between a large number of test samples of which concentration is not known can be calculated with high accuracy without conversion of fluorescence equivalent to an amplification amount of the template to molar concentration of dsDNA and without standard curve using multiple standard samples of which concentrations are known. In addition, use of at least one of the standard sample of which the initial template concentration is known makes possible calculation of the initial template amounts of a large number of test samples of which concentrations are not known.

### Other Embodiments

The embodiments of the present invention have been described above. However, the present invention may be executed in not only the embodiments described above but also various different embodiments within the technical idea described in the scope of the invention.

In the above embodiments, an example in which the target nucleic acid measuring apparatus 100 mainly performs the processes in a standalone mode is explained. However, as described in the embodiments, a process may be performed in response to a request from another terminal apparatus constituted by a housing different from that of the target nucleic acid measuring apparatus 100, and the process result may be returned to the client terminal.

Of each of the processes explained in the embodiments, all or some processes explained to be automatically performed may be manually performed. Alternatively, all or some processes explained to be manually performed may also be automatically performed by a known method. For example, a target nucleic acid measuring method as follows may be executed by a control unit.
(Note 1) A target nucleic acid measuring method of calculating an amount equivalent to an initial template amount in a test sample by fitting a theoretical expression to an amount equivalent to an amplification amount of target nucleic acid for each thermal cycle number,

wherein the theoretical expression includes an environmental coefficient as an exponent parameter, and a parameter of the amount equivalent to the initial template amount, and includes at least one parameter among an amount equivalent to a saturation amount upon the target nucleic acid amplification, a reaction acceleration coefficient, and a reaction inhibition coefficient.
(Note 2) The target nucleic acid measuring method according to Note 1,

wherein an amplification efficiency of the nucleic acid amplification reaction is defined by the environmental coefficient and at least one parameter among the amount equivalent to the saturation amount upon the target nucleic acid amplification, the reaction acceleration coefficient, and the reaction inhibition coefficient, in the theoretical expression.
(Note 3) The target nucleic acid measuring method according to Note 2,

wherein the theoretical expression expresses that the amount equivalent to the amplification amount is equal to the product of the initial template amount and a sequence of the amplification efficiencies to each of which 1 is added for each thermal cycle number.
(Note 4) The target nucleic acid measuring method according to Note 3,

wherein the theoretical expression is represented by the following expression:

$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$

(wherein Nₙ is the amount equivalent to the amplification amount of the target nucleic acid at thermal cycle number n; N₀ is the amount equivalent to the initial template amount; j is the thermal cycle number; Nⱼ is the amount equivalent to the amplification amount of the target nucleic acid at the thermal cycle number j; Nₘₐₓ is the amount equivalent to the saturation amount upon the target nucleic acid amplification; ρ is the reaction acceleration coefficient; μ is the reaction inhibition coefficient; and K is the environmental coefficient).
(Note 5) The target nucleic acid measuring method according to any one of Notes 1 to 4,

wherein the theoretical expression is fitted using the least square method.
(Note 6) The target nucleic acid measuring method according to any one of Notes 1 to 4,

wherein at least one of the reaction acceleration coefficient, the reaction inhibition coefficient, the environmental coefficient, and the amount equivalent to the saturation amount upon the target nucleic acid amplification in the theoretical expression is a fixed value.
(Note 7) The target nucleic acid measuring method according to Note 6,

wherein any one of the reaction acceleration coefficient and the reaction inhibition coefficient or both are calculated to be fixed by fitting the theoretical expression to the amount equivalent to the amplification amount in a region where an increasing rate represented by the following expression is from 0 to 1.

$${Δ}_{n} = \frac{{N}_{n}}{{N}_{n - 1}} - 1$$

(wherein, Δₙ is the increasing rate; and Nₙ is the amount equivalent to the amplification amount of the target nucleic acid at thermal cycle number n.)
(Note 8) The target nucleic acid measuring method according to Note 6,

wherein the maximum value of the amount equivalent to the amplification amount of the target nucleic acid is set as the amount equivalent to the saturation amount upon the target nucleic acid amplification.
(Note 9) The target nucleic acid measuring method according to any one of Notes 1 to 8,

wherein the calculated amount equivalent to the initial template amount is converted to a value indicating the initial template amount.
(Note 10) The target nucleic acid measuring method according to Note 9,

wherein the amount equivalent to the amplification amount of the target nucleic acid is a measurement value of a signal from an intercalator that is added to the test sample and emits the signal only in the presence of a double-strand DNA but not in the absence of the double-strand DNA,

and wherein the amount equivalent to the initial template amount is converted to the value indicating the initial template amount at the following steps:

a) a step of performing a nucleic acid amplification reaction of a standard sample having the same target nucleic acid as the test sample under the same condition as the test sample, measuring an amplification amount of the amplification product of the target nucleic acid, and preparing a dilution series of the amplification product to which the intercalator is added under the same conditions as the test sample;

b) a step of measuring the signal from the intercalator in the dilution series prepared at the step a) under the same conditions as the test sample to obtain the measurement value;

c) a step of determining the relational expression between the measurement value obtained at the step b) and the concentration of the dilution series prepared at the step a); and

d) a step of converting the amount equivalent to the initial template amount to the value indicating the initial template amount based on the relational expression determined at the step c).
(Note 11) The target nucleic acid measuring method according to Note 9,

wherein the amount equivalent to the amplification amount of the target nucleic acid is a measurement value of a signal from a reporter molecule that is added to the test sample and emits the signal according to an extension reaction during the nucleic acid amplification reaction,

and wherein the amount equivalent to the initial template amount is converted to the value indicating the initial template amount at the following steps:

a) a step of preparing a dilution series of a fluorescent molecule that emits the same signal as the reporter molecule;

b) a step of measuring the signal from the fluorescent molecule in the dilution series prepared at the step a) under the same conditions as the test sample to obtain the measurement value;

c) a step of determining the relational expression between the measurement value obtained at the step b) and the concentration of the dilution series prepared at the step a); and

d) a step of converting the amount equivalent to the initial template amount to the value indicating the initial template amount based on the relational expression determined at the step c).
(Note 12) The target nucleic acid measuring method according to Note 9,

wherein the amount equivalent to the amplification amount of the target nucleic acid is a measurement value of a signal from a reporter molecule that is added to the test sample and emits the signal according to an extension reaction during the nucleic acid amplification reaction,

and wherein the amount equivalent to the initial template amount is converted to the value indicating the initial template amount at the following steps:

a) a step of performing a nucleic acid amplification reaction of a standard sample having the reporter molecule and the same target nucleic acid as the test sample under the same condition as the test sample, measuring an amplification amount of the amplification product of the target nucleic acid, and preparing a dilution series of the amplification product;

b) a step of measuring the signal from the reporter molecule in the dilution series prepared at the step a) under the same conditions as the test sample to obtain the measurement value;

c) a step of determining the relational expression between the measurement value obtained at the step b) and the concentration of the dilution series prepared at the step a); and

d) a step of converting the amount equivalent to the initial template amount to the value indicating the initial template amount based on the relational expression determined at the step c)
(Note 13) The target nucleic acid measuring method according to any one of Notes 1 to 8,

wherein multiple test samples having the same target nucleic acid are subjected to the nucleic acid amplification reaction,

and wherein a relation of the initial template amount between the test samples is calculated by comparing the amounts equivalent to the initial template amount calculated for each of the test samples.
(Note 14) The target nucleic acid measuring method according to Note 13,

wherein at least one of the test samples is a standard sample of which the initial template amount is known,

and wherein a ratio of the amount equivalent to the initial template amount of each of the test samples to that of the standard sample is calculated as the relation of the initial template amount between the test samples, and the initial template amount for each of the test samples is calculated by multiplying the calculated ratio by the initial template amount of the standard sample.
(Note 15) The target nucleic acid measuring method according to Note 14,

wherein at least one of the parameters in the theoretical expression fitted to the amount equivalent to the amplification amount of the target nucleic acid for the standard sample for each thermal cycle number is fixed to calculate the initial template amount for each of the test samples.

In addition, the procedures, the control procedures, the specific names, the information including parameters such as registered data or search condition, examples of screen image and the database configurations which are described in the literatures or the drawings may be arbitrarily changed unless otherwise noted.

With respect to the target nucleic acid measuring apparatus 100, the constituent elements shown in the drawings are functionally schematic. The constituent elements need not be always physically arranged as shown in the drawings.

For example, in the embodiment described above, the measuring unit 116 was constituted as one component included in the target nucleic acid measuring apparatus 100, but the present invention is not intended to be limited thereto. For example, the measuring unit 116 may be replaced with a measuring apparatus such as a real-time PCR apparatus, including a measuring unit that measures an amount equivalent to an amplification amount of target nucleic acid corresponding to each thermal cycle number in a nucleic acid amplification reaction. That is, the invention may be constituted as a target nucleic acid measuring system including a measuring apparatus with a measuring unit, and an information processing apparatus with a control unit and a storage unit. In this target nucleic acid measuring system, the measuring unit of the measuring apparatus has the same function as the measuring unit 116 according to the above embodiments, and the storage unit and the control unit of the information processing apparatus has the same function as the storage unit 106 and the control unit 102 of the target nucleic acid measuring apparatus 100 according to the above embodiments, and thus the system is constituted so as to have the similar effect. An example of the target nucleic acid measuring system is as follows.

A target nucleic acid measuring system comprising a measuring apparatus with a measuring unit that measures an amount equivalent to an amplification amount of target nucleic acid at each thermal cycle number, and an information processing apparatus with a storage unit and a control unit,

wherein the storage unit includes:

an amplification amount equivalent storage unit that stores the amount equivalent to the amplification amount of the target nucleic acid, which is measured by the measuring apparatus corresponding to thermal cycle number; and

a theoretical expression storage unit that stores a theoretical expression including an environmental coefficient as an exponent parameter, and a parameter of the amount equivalent to the initial template amount in the test sample, and including at least one parameter among an amount equivalent to a saturation amount upon the target nucleic acid amplification, a reaction acceleration coefficient, and a reaction inhibition coefficient,

and wherein the control unit includes:

a theoretical expression fitting unit that fits the theoretical expression to the amount equivalent to the amplification amount of the target nucleic acid for each thermal cycle number stored in the storage unit; and

an initial template amount equivalent calculating unit that calculates the amount equivalent to the initial template amount from the theoretical expression fitted by the theoretical expression fitting unit.

In addition, all or some processing functions of the devices in the target nucleic acid measuring apparatus 100, in particular, processing functions performed by the control unit 102 may be realized by a central processing unit (CPU) and a program interpreted and executed by the CPU or may also be realized by hardware realized by a wired logic. The program is recorded on a recording medium (will be described later) and mechanically read by the target nucleic acid measuring apparatus 100 as needed. More specifically, on the storage unit 106 such as a ROM or an HD, a computer program which gives an instruction to the CPU in cooperation with an operating system (OS) to perform various processes is recorded. The computer program is executed by being loaded on a RAM, and constitutes a control unit in cooperation with the CPU. As an example of a target nucleic acid measuring program, the following may be utilized.

A computer program product having a computer readable medium including programmed instructions for a computer having a control unit and a storage unit,

wherein the storage unit includes:

an amplification amount equivalent storage unit that stores an amount equivalent to an amplification amount of target nucleic acid, corresponding to thermal cycle number; and

a theoretical expression storage unit that stores a theoretical expression including an environmental coefficient as an exponent parameter, and a parameter of an amount equivalent to an initial template amount in the test sample, and including at least one parameter among an amount equivalent to a saturation amount upon the target nucleic acid amplification, a reaction acceleration coefficient, and a reaction inhibition coefficient, and

wherein the instructions, when executed by the control unit of the computer, cause the computer to perform:

a theoretical expression fitting step of fitting the theoretical expression to the amount equivalent to the amplification amount of the target nucleic acid for each thermal cycle number stored in the storage unit; and

an initial template amount equivalent calculating step of calculating the amount equivalent to the initial template amount from the theoretical expression fitted at the theoretical expression fitting step.

The computer program may be stored in an application program server connected to the target nucleic acid measuring apparatus 100 through an arbitrary network 300. The computer program in whole or in part may be downloaded as needed.

A program which causes a computer to execute a method according to the present invention may also be stored in a computer readable recording medium. In this case, the “recording medium” includes an arbitrary “portable physical medium” such as a flexible disk, a magnet-optical disk, a ROM, an EPROM, an EEPROM, a CD-ROM, an MO, or a DVD or a “communication medium” such as a communication line or a carrier wave which holds a program for a short period of time when the program is transmitted through a network typified by a LAN, a WAN, and the Internet.

The “program” is a data processing method described in an arbitrary language or a describing method. As a format of the “program”, any format such as a source code or a binary code may be used. The “program” is not always singularly constructed, and includes a program obtained by distributing and arranging multiple modules or libraries or a program that achieves the function in cooperation with another program typified by an operating system (OS). In the apparatuses according to the embodiments, as a specific configuration to read a recording medium, a read procedure, an install procedure used after the reading, and the like, known configurations and procedures may be used.

Various databases or the like (the measurement data file 106a, the theoretical expression file 106b, the relational expression file 106c and the like) stored in the storage unit 106 are a memory device such as a RAM or a ROM, a fixed disk device such as a hard disk drive, and a storage unit such as a flexible disk or an optical disk and store various programs, tables, databases, Web page files used in various processes or Web site provision.

The target nucleic acid measuring apparatus 100 may be realized by connecting a known information processing apparatus such as a personal computer or a workstation and installing software (including a program, data, or the like) which causes the information processing apparatus to realize the method according to the present invention.

Furthermore, a specific configuration of distribution and integration of the devices is not limited to that shown in the drawings. All or some devices can be configured such that the devices are functionally or physically distributed and integrated in arbitrary units depending on various additions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A target nucleic acid measuring apparatus comprising a storage unit and a control unit,
wherein the storage unit includes:
an amplification amount equivalent storage unit that stores an amount equivalent to an amplification amount of target nucleic acid, corresponding to thermal cycle number; and
a theoretical expression storage unit that stores a theoretical expression including an environmental coefficient as an exponent parameter, and a parameter of an amount equivalent to an initial template amount in the test sample, and including at least one parameter among an amount equivalent to a saturation amount upon the target nucleic acid amplification, a reaction acceleration coefficient, and a reaction inhibition coefficient, and
wherein the control unit includes:
a theoretical expression fitting unit that fits the theoretical expression to the amount equivalent'to the amplification amount of the target nucleic acid for each thermal cycle number stored in the storage unit; and
an initial template amount equivalent calculating unit that calculates the amount equivalent to the initial template amount from the theoretical expression fitted by the theoretical expression fitting unit.

2. The target nucleic acid measuring apparatus according to claim 1,
wherein an amplification efficiency of the nucleic acid amplification reaction is defined by the environmental coefficient and at least one parameter among the amount equivalent to the saturation amount upon the target nucleic acid amplification, the reaction acceleration coefficient, and the reaction inhibition coefficient, in the theoretical expression stored in the theoretical expression storage unit.

3. The target nucleic acid measuring apparatus according to claim 2,
wherein the theoretical expression stored in the theoretical expression storage unit, expresses that the amount equivalent to the amplification amount of the target nucleic acid is equal to the product of the amount equivalent to the initial template amount and a sequence of the amplification efficiencies to each of which 1 is added for each thermal cycle number.

4. The target nucleic acid measuring apparatus according to claim 3,
wherein the theoretical expression stored in the theoretical expression storage unit is represented by the following expression:
$${N}_{n} = {N}_{0} {\prod }_{j = 1}^{n} \{ 1 + {ρ ( \frac{{N}_{max} - {N}_{j - 1}}{{N}_{max} + μ {N}_{j - 1}} )}^{K} \}$$
(wherein Nₙ is the amount equivalent to the amplification amount of the target nucleic acid at thermal cycle number n; N₀ is the amount equivalent to the initial template amount; j is the thermal cycle number; Nⱼ is the amount equivalent to the amplification amount of the target nucleic acid at the thermal cycle number j; Nₘₐₓ is the amount equivalent to the saturation amount upon the target nucleic acid amplification; ρ is the reaction acceleration coefficient; μ is the reaction inhibition coefficient; and K is the environmental coefficient).

5. The target nucleic acid measuring apparatus according to claim 1,
wherein the theoretical expression fitting unit fits the theoretical expression using the least square method.

6. The target nucleic acid measuring apparatus according to claim 1,
wherein the theoretical expression storage unit stores the theoretical expression in which at least one of the reaction acceleration coefficient, the reaction inhibition coefficient, the environmental coefficient, and the amount equivalent to the saturation amount upon the target nucleic acid amplification is a fixed value.

7. The target nucleic acid measuring apparatus according to claim 6,
wherein the theoretical expression fitting unit calculates and fixes any one of the reaction acceleration coefficient and the reaction inhibition coefficient or both by fitting the theoretical expression to the amount equivalent to the amplification amount in a region where an increasing rate represented by the following expression is from 0 to 1.
$${Δ}_{n} = \frac{{N}_{n}}{{N}_{n - 1}} - 1$$
(wherein, Δₙ is the increasing rate; and Nₙ is the amount equivalent to the amplification amount of the target nucleic acid at thermal cycle number n.)

8. The target nucleic acid measuring apparatus according to claim 6,
wherein the theoretical expression fitting unit sets the maximum value of the amount equivalent to the amplification amount of the target nucleic acid as the amount equivalent to the saturation amount upon the target nucleic acid amplification.

9. The target nucleic acid measuring apparatus according to claim 1,
wherein the control unit further includes an initial template amount value converting unit that converts the amount equivalent to the initial template amount calculated by the initial template amount equivalent calculating unit, to a value indicating the initial template amount.

10. The target nucleic acid measuring apparatus according to claim 9,
wherein the amount equivalent to the amplification amount of the target nucleic acid stored in the amplification amount equivalent storage unit, is a measurement value of a signal from an intercalator that is added to the test sample and emits the signal only in the presence of a double-strand DNA but not in the absence of the double-strand DNA,
and wherein the initial template amount value converting unit converts the amount equivalent to the initial template amount, to the value indicating the initial template amount based on a relational expression determined at the following steps:
a) a step of performing a nucleic acid amplification reaction of a standard sample having the same target nucleic acid as the test sample under the same condition as the test sample, measuring an amplification amount of the amplification product of the target nucleic acid, and preparing a dilution series of the amplification product to which the intercalator is added under the same conditions as the test sample;
b) a step of measuring the signal from the intercalator in the dilution series prepared at the step a) under the same conditions as the test sample to obtain the measurement value; and
c) a step of determining the relational expression between the measurement value obtained at the step b) and the concentration of the dilution series prepared at the step a).

11. The target nucleic acid measuring apparatus according to claim 9,
wherein the amount equivalent to the amplification amount of the target nucleic acid stored in the amplification amount equivalent storage unit, is a measurement value of a signal from a reporter molecule that is added to the test sample and emits the signal according to an extension reaction during the nucleic acid amplification reaction,
and wherein the initial template amount value converting unit converts the amount equivalent to the initial template amount, to the value indicating the initial template amount based on a relational expression determined at the following steps:
a) a step of preparing a dilution series of a fluorescent molecule that emits the same signal as the reporter molecule;
b) a step of measuring the signal from the fluorescent molecule in the dilution series prepared at the step a) under the same conditions as the test sample to obtain the measurement value; and
c) a step of determining the relational expression between the measurement value obtained at the step b) and the concentration of the dilution series prepared at the step a).

12. The target nucleic acid measuring apparatus according to claim 9,
wherein the amount equivalent to the amplification amount of the target nucleic acid stored in the amplification amount equivalent storage unit, is a measurement value of a signal from a reporter molecule that is added to the test sample and emits the signal according to an extension reaction during the nucleic acid amplification reaction,
and wherein the initial template amount value converting unit converts the amount equivalent to the initial template amount, to the value indicating the initial template amount based on a relational expression determined at the following steps:
a) a step of performing a nucleic acid amplification reaction of a standard sample having the reporter molecule and the same target nucleic acid as the test sample under the same condition as the test sample, measuring an amplification amount of the amplification product of the target nucleic acid, and preparing a dilution series of the amplification product;
b) a step of measuring the signal from the reporter molecule in the dilution series prepared at the step a) under the same conditions as the test sample to obtain the measurement value; and
c) a step of determining the relational expression between the measurement value obtained at the step b) and the concentration of the dilution series prepared at the step a).

13. The target nucleic acid measuring apparatus according to claim 1,
wherein the amplification amount equivalent storage unit stores the amount equivalent to the amplification amount of the target nucleic acid corresponding to the thermal cycle number for each of multiple test samples having the same target nucleic acid,
and wherein the control unit further includes an initial template amount relation calculating unit that calculates a relation of the initial template amount between the test samples by comparing the amounts equivalent to the initial template amount calculated by the initial template amount equivalent calculating unit for each of the test samples.

14. The target nucleic acid measuring apparatus according to claim 13,
wherein at least one of the test samples stored in the amplification amount equivalent storage unit, is a standard sample of which the initial template amount is known,
and wherein the initial template amount relation calculating unit calculates a ratio of the amount equivalent to the initial template amount of each of the test samples to that of the standard sample as the relation of the initial template amount between the test samples, and multiplies the calculated ratio by the initial template amount of the standard sample to calculate the initial template amount for each of the test samples.

15. The target nucleic acid measuring apparatus according to claim 14,
wherein the theoretical expression fitting unit fixes at least one of the parameters in the theoretical expression fitted to the amount equivalent to the amplification amount of the target nucleic acid for the standard sample for each thermal cycle number to calculate the initial template amount for each of the test samples.
